(19) United States
(12) Patent Application Publication
(10) Pub. No.: US 2009/0074695 A1
(43) Pub. Date: Mar. 19, 2009

(54) COSMETIC COMPOSITIONS COMPRISING AT LEAST ONE CATIONIC COPOLYMER, AT LEAST ONE AMINO SILICONE AND AT LEAST ONE CATIONIC POLYMER, AND METHODS OF USE THEREOF

(76) Inventors: Veronique MAHE, Vaux S/Seine (FR); Olga BIGANSKA, Asnieres sur Seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/210,607

(22) Filed: Sep. 15, 2008

Related U.S. Application Data
(60) Provisional application No. 60/960,278, filed on Sep. 24, 2007.

(30) Foreign Application Priority Data
Sep. 14, 2007 (FR) 0757579

(51) Int. Cl. A61K 8/81; A61Q 5/12; A61Q 5/00; A61Q 5/02

(52) U.S. Cl. 424/70.11; 510/122

(57) ABSTRACT

The present disclosure relates to novel cosmetic compositions comprising, in a cosmetically acceptable medium:
(i)—at least one cationic polymer, which is a product of polymerization of a monomer mixture comprising:
   a) at least one vinyl monomer substituted with at least one amino group, and
   b) at least one hydrophobic nonionic vinyl monomer chosen from formula (I) and (II):

CH₂═C(X)Z, and (I)

CH₂═CH—OC(O)R; (II)
wherein:
X is chosen from a hydrogen atom and a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R independently is chosen from a C₁-C₃₀ alkyl group;
each R¹ independently is chosen from a C₁-C₃₀ alkyl group, a C₂-C₃₀ hydroxyalkyl group, and a C₁-C₃₀ haloalkyl group; and
   c) at least one associative vinyl monomer,
(ii)—at least one amino silicone, and
(iii)—at least one cationic polymer other than the at least one cationic polymer (i) and the at least one amino silicone (ii) and having a cationic charge density of greater than or equal to 4 meq./g.

These compositions may be used for washing and/or conditioning keratin materials such as the hair or the skin.

This application claims benefit of U.S. Provisional Application No. 60/960,278, filed Sep. 24, 2007, the contents of which are incorporated herein by reference. This application also claims benefit of priority under 35 U.S.C. § 119 to French Patent Application No. FR 0757579, filed Sep. 14, 2007, the contents of which are also incorporated herein by reference.

The present disclosure relates to novel cosmetic compositions comprising, in a cosmetically acceptable medium, at least one cationic copolymer, at least one amino silicone, and at least one cationic polymer.

It is well known that hair which has been sensitized (i.e., damaged and/or embrittled) to varying degrees by the action of atmospheric agents or the action of mechanical or chemical treatments, such as dyes, bleaches, and/or permanent-waving, is often difficult to disentangle and to style, and lacks softness.

It is known to use conditioners, for example, cationic and amphoteric polymers or silicones, in compositions for washing or caring for keratin material such as the hair, in order to disentangle the hair and to give it softness and flexibility. However, these polymers or silicones may have the disadvantage of lankness of the hairstyle (lack of lightness of the hair) and lack of smoothness (hair not uniform from the root to the end) on dried hair.

In addition, the use of cationic or amphoteric polymers for this purpose has other drawbacks. On account of their high affinity for the hair, some of these polymers become deposited thereon to a large extent during repeated use, and lead to adverse effects such as an unpleasant, laden feel, stiffening of the hair, and interfiber adhesion which has an effect on styling. These drawbacks are accentuated in the case of fine hair, which lacks liveliness and body.

It is also known in the hair field to use amino silicones in shampoo compositions as conditioning agents for improving the softness, feel and disentangling of the hair. However, it has been found that these silicones lead to the formation of an unattractive layer at the surface of the shampoo, which is harmful to the performance of the shampoo. To avoid the appearance of this phenomenon, stabilizers such as crosslinked acrylic polymers of the CARBOPOL® 980 type are frequently used. However, these stabilizers have the drawback of reducing the cosmetic performance of shampoos, for example, by making the hair more laden and more coarse.

Dialkyldiallylammonium polymers are often used in shampoo compositions to improve the cosmetic properties of sensitized hair, for example, to make it less coarse and/or to improve its disentangling capacity.

However, it has been observed that these dialkyldiallylammonium polymers often have the drawback of reducing the cosmetic performance of natural hair, for instance, by making it lank.

Accordingly, there is a need to develop a detergent cosmetic composition, such as a shampoo, which can overcome such disadvantages found with current cosmetic compositions comprising cationic polymers and improve cosmetic performance on keratin materials, i.e., the hair and the scalp, such as natural hair.

Such detergent compositions, comprising a cationic copolymer as an agent for stabilizing or suspending water-insoluble ingredients such as silicones or fatty substances have been described for example, in International Patent Application Publication No. WO 2005/092276. However, the foam qualities and the cosmetic properties obtained with these compositions are still not sufficiently satisfactory.

The present inventors have discovered that the combination of a at least one cationic copolymer, at least one second cationic polymer, and at least one silicone makes it possible to overcome these drawbacks.

Specifically, it has been found that the use of the at least one cationic copolymer in the compositions disclosed herein produces on keratin materials, such as the hair, very good cosmetic properties, for instance lightness, softness, smooth feel, suppleness, and manageability of dried hair. It has also been found with the compositions disclosed herein, dried hair may look, for example, smoother.

Moreover, the compositions according to the present disclosure are stable and have an attractive visual appearance, for example consistency, abundance of foam and elimination of foam.

The compositions, as disclosed herein, when applied to the skin, such as in the form of a bubble bath or a shower gel, may improve the softness of the skin.

Thus, one aspect of the present disclosure is a novel cosmetic composition comprising, in a cosmetically acceptable medium:

(i)—at least one cationic polymer produced by polymerization of a monomer mixture comprising:
a) at least one vinyl monomer substituted with at least one amino group,
b) at least one hydrophobic nonionic vinyl monomer chosen from formula (I) and (II):

CH₂═C(X)Z, (1)

CH₂═CH—OC(O)R; (II)

wherein:

X is chosen from a hydrogen atom and a methyl group;

Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;

x is an integer ranging from 1 to 6;

each R independently is a C₁-C₃₀ alkyl group;

each R¹ independently is chosen from a C₁-C₃₀ alkyl group, a C₂-C₃₀ hydroxyalkyl group, and a C₁-C₃₀ haloalkyl group, and
c) at least one associative vinyl monomer,

(ii)—at least one amino silicone, and

(iii)—at least one cationic polymer other than the at least one cationic polymer (i) and the at least one amino silicone (ii) and having a cationic charge density of greater than or equal to 4 meq./g.

Another aspect of the present disclosure is a cosmetic composition and use thereof for giving the hair one or more properties chosen from sheen, lightness, softness, a smooth feel, and/or suppleness.

Yet another aspect of the present disclosure is a process for treating keratin materials, such as the hair, comprising applying the cosmetic composition as disclosed herein to the keratin materials.

As used herein, “keratin materials” is understood to mean the hair, the eyelashes, the eyebrows, the skin, the nails, mucous membranes, or the scalp, for instance, the hair.

Another aspect of the present disclosure is the use of at least one cationic copolymer (i) as disclosed herein, or for the manufacture of, a cosmetic composition comprising at least one cationic polymer (iii) other than the at least one cationic polymer (i) and having a cationic charge density of greater than or equal to 4 meq./g and at least one amino silicone.

One embodiment of the present disclosure is a cationic polymer obtained by polymerization of a monomer mixture comprising at least one vinyl monomer substituted with at least one amino group, at least one hydrophobic nonionic vinyl monomer, and at least one associative vinyl monomer. In at least one embodiment, the monomers constituting the cationic copolymer also comprise at least one semi-hydrophobic vinyl surfactant monomer. The various monomers of the polymers as disclosed herein are different from each other.

For example, the at least one cationic polymer (i) is a thickening polymer.

As disclosed herein, “thickening polymer” is understood to mean a polymer which, when introduced at 1% by weight into an aqueous or aqueous-alcoholic solution comprising 30% by weight of ethanol, and at pH 7, makes it possible to achieve a viscosity of at least 100 cps at 25° C., and at a shear rate of 1 s⁻¹. This viscosity may be measured using a viscometer with cone-plate geometry, for example a Haake RS 600 rheometer. In at least one embodiment, these polymers make it possible to increase the viscosity of the compositions in which they are present by at least 50 cps at 25° C. and at 1 s⁻¹.

The at least one cationic polymer (i) used in the composition disclosed herein, and the process for manufacturing them, are described, for example, in International Patent Application Publication No. WO 2004/024779.

As disclosed herein, “vinyl monomer” is understood to mean a monomer comprising at least one R₀CH═C(R₀)— group, wherein each R₀ is independently chosen from a hydrogen atom, C₁-C₃₀ alkyl, —C(O)OH, C(O)OR₀′, —O—C(O)OR₀′, —C(O)NHR₀′, —C(O)NHR₀′, and C(O)NR₀′R₀″, wherein R₀′ and R₀″, which may be identical or different, are chosen from C₁-C₃₀ alkyl groups. Such vinyl monomers include, for example, (meth)acrylates and (meth)acrylamides.

According to the present disclosure, the monomer mixture for preparing the at least one cationic polymer (i) used in the composition comprises at least one vinyl monomer substituted with at least one amino group.

The at least one vinyl monomer substituted with at least one amino group that may be used for the preparation of the at least one cationic polymer (i) as disclosed herein, is chosen from basic, polymerizable ethylenically unsaturated monomers. The at least one amine group may be derived from monoamino, diamino, or polyamino alkyl groups, or from heteroaromatic groups comprising a nitrogen atom. The at least one amine group may also be chosen from a primary, secondary, and tertiary amine. These monomers may be used in the form of amine or in the form of salt.

In at least one embodiment, the at least one vinyl monomer substituted with the at least one amine group is chosen from:

mono(C₁-C₄)alkylamino(C₁-C₈)alkyl (meth)acrylates,
di(C₁-C₄)alkylamino(C₁-C₈)alkyl (meth)acrylates, such as di(C₁-C₄)alkylamino(C₁-C₆)alkyl (meth)acrylates,

mono(C₁-C₄)alkylamino(C₁-C₈)alkyl(meth)acrylamides,

di(C₁-C₄)alkylamino(C₁-C₈)alkyl(meth)acrylamides,

(meth)acrylamides with at least one heterocyclic group comprising a nitrogen atom,

(meth)acrylates with at least one heterocyclic group comprising a nitrogen atom

nitrogenous heterocycles comprising at least one vinyl group,

and mixtures thereof.

Non-limiting examples of vinyl monomers substituted with at least one amino group that may be mentioned include:
mono- or di(C₁-C₄ alkyl)amino(C₁-C₄ alkyl) (meth)acrylates, such as 2-(N,N-dimethylamino)ethyl (meth)acrylate, 3-(N,N-dimethylamino)propyl (meth)acrylate, 4-(N,N-dimethylamino)butyl (meth)acrylate, (N,N-dimethylamino)-t-butyl (meth)acrylate, 2-(N,N-diethylamino)ethyl (meth)acrylate, 3-(N,N-diethylamino)propyl (meth)acrylate, 4-(N,N-diethylamino)butyl (meth)acrylate, 2-(N,N-dipropylamino)ethyl (meth)acrylate, 3-(N,N-dipropylamino)propyl (meth)acrylate, and 4-(N,N-dipropylamino)butyl (meth)acrylate;

mono- or di(C₁-C₄ alkyl)amino(C₁-C₄ alkyl)(meth)acrylamides, such as N′-(2-N,N-dimethylamino)ethyl(meth)acrylamide and N′-(3-N,N-dimethylamino)propylacrylamide;

(meth)acrylamides or (meth)acrylates with a heterocyclic group comprising a nitrogen atom, such as N-(2-pyridyl)acrylamide, N-(2-imidazolyl)methacrylamide, 2-(4-morpholinyl)ethyl methacrylate, 2-(4-morpholinyl)ethyl acrylate, N-(4-morpholinyl)-methacrylamide, and N-(4-morpholinyl)acrylamide; and

nitrogenous heterocycles comprising at least one vinyl group, such as 2-vinylpyridine and 4-vinylpyridine,

When the monomers are in the form of salts, they may be mineral salts, such as hydrochloride, sulfate, and phosphate salts; or organic acid salts, such as acetate, maleate, and fumarate salts

Among the vinyl monomers substituted with at least one amino group that may be mentioned include, but are not limited to:
3-(N,N-dimethylamino)propyl (meth)acrylate,
N′-(3-N,N-dimethylamino)propyl(meth)acrylamide,
2-(N,N-dimethylamino)ethyl (meth)acrylate,
2-(N,N-diethylamino)ethyl (meth)acrylate,
2-(tert-butylamino)ethyl (meth)acrylate,
2-(N,N-dimethylamino)propyl(meth)acrylamide, and
2-(N,N-dimethylamino)neopentyl acrylate.

The at least one vinyl monomer substituted with at least one amino group may be present in an amount ranging from 10% to 70% by weight, for example, from 20% to 60% by weight and further, for example, from 30% to 40% by weight, relative to the total weight of the monomer mixture.

According to the present disclosure, the monomer mixture for preparing the at least one cationic polymer (i) also comprises at least one hydrophobic nonionic vinyl monomer b).

The at least one hydrophobic nonionic vinyl monomer b) for the preparation of the at least one cationic polymer disclosed herein is, in at least one embodiment, chosen from compounds of formulae (I) and (II):

CH₂═C(X)Z, (I)

CH₂═CH—OC(O)R; (II)

wherein:

X is chosen from a hydrogen atom and a methyl group;

Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;

x is an integer ranging from 1 to 6;

each R independently is a C₁-C₃₀ alkyl group;

each R¹ independently is a C₁-C₃₀ alkyl group, a C₂-C₃₀ hydroxyalkyl group and a C₁-C₃₀ haloalkyl group.

Non-limiting mention may be made, for example, of C₁-C₃₀ alkyl (meth)acrylates; (C₁-C₃₀ alkyl)(meth)acrylamides; styrene, substituted styrenes, such as as vinyltoluene (or 2-methylstyrene), butylstyrene, isopropylstyrene, and para-chlorostyrene; vinyl esters, such as vinyl acetate, vinyl butyrate, vinyl caprylate, vinyl pidolate, and vinyl neodecanoate; unsaturated nitrites, for example (meth)acrylonitrile and acrylonitrile; and unsaturated silanes, for instance trimethylvinylsilane, dimethylethylvinylsilane, allyidimethylphenylsilane, allyltrimethylsilane, 3-acrylamidopropyltrimethylsilane, and 3-trimethylsilylpropyl methacrylate.

According to at least one embodiment, the at least one hydrophobic nonionic vinyl monomer is chosen from C₁-C₃₀ alkyl acrylates, C₁-C₃₀ alkyl methacrylates, and mixtures thereof, such as ethyl acrylate, methyl methacrylate, and 3,3,5-trimethylcyclohexyl methacrylate.

The at least one hydrophobic nonionic vinyl monomer may be present in an amount ranging from 20% to 80% by weight, such as from 20% to 70% by weight, and further such as from 50% to 65% by weight, relative to the total weight of the monomer mixture.

The at least one associative vinyl monomer that may be used for the preparation of the at least one cationic polymer (i) according to the present disclosure may be chosen, for example, from compounds having an ethylenically unsaturated end (i)′ for addition polymerization with other monomers of the system, a polyoxyalkylene central portion (ii)′ for giving the polymers selective hydrophilic properties, and a hydrophobic end (iii)′ for giving the polymers selective hydrophobic properties.

The ethylenically unsaturated end (i)′ of the at least one associative vinyl monomer is derived, for example, from an α,β-ethylenically unsaturated monocarboxylic or dicarboxylic acid or anhydride, such as a C₃ or C₄ monocarboxylic or dicarboxylic acid or anhydride. Alternatively, the end (i)′ of the associative monomer may be derived from an allyl ether or a vinyl ether; from a nonionic urethane monomer substituted with a vinyl group, as described in U.S. Pat. No. Re. 33,156 or in U.S. Pat. No. 5,294,692; or a product of reaction of urea substituted with a vinyl group, as described in U.S. Pat. No. 5,011,978.

The central portion (ii)′ of the at least one associative vinyl monomer is, for example, a polyoxyalkylene segment comprising 5 to 250 C₂-C₇ alkylene oxide units, such as 10 to 120 C₂-C₇ alkylene oxide units and further such as 15 to 60 C₂-C₇ alkylene oxide units. In at least one embodiment, central portions (ii)′ are polyoxyethylene, polyoxypropylene, and polyoxybutylene segments comprising 5 to 150, such as 10 to 100, and further such as 15 to 60, ethylene oxide, propylene oxide, or butylene oxide units, and random or non-random blocks of ethylene oxide, propylene oxide, or butylene oxide units. In another embodiment, the central portions (ii)′ are polyoxyethylene segments.

The hydrophobic end (iii)′ of the at least one associative monomer is, in at least one embodiment, a hydrocarbon-based fragment belonging to one of the following hydrocarbon classes: a linear alkyl, a C₂-C₄₀ alkyl substituted with an aryl group, a phenyl substituted with a C₂-C₄₀ alkyl group, a branched alkyl, an alicyclic group, and a complex ester.

As used herein, “complex ester” means any ester other than a simple ester.

As used herein, “simple ester” means any ester of an unsubstituted, linear or branched saturated C₁-C₃₀ aliphatic alcohol.

Non-limiting examples of the hydrophobic ends (iii)′ of the at least one associative monomer include linear and branched C₈-C₄₀ alkyl groups, such as capryl (C₈), isooctyl (branched C₈), decyl (C₁₀), lauryl (C₁₂), myristyl (C₁₄), cetyl (C₁₆), cetearyl (C₁₆-C₁₈), stearyl (C₁₈), isostearyl (branched C₁₈), arachidyl (C₂₀), behenyl (C₂₂), lignoceryl (C₂₄), cerotyl (C₂₆), montanyl (C₂₈), melissyl (C₃₀), and lacceryl (C₃₂) groups.

Non-limiting examples of linear and branched C₈-C₄₀ alkyl groups derived from a natural source include alkyl groups derived from hydrogenated groundnut oil, soybean oil and canola oil (predominantly C₁₈), C₁₆-C₁₈ hydrogenated tallow oil; and C₁₀-C₃₀ hydrogenated terpenols, such as hydrogenated geraniol (branched C₁₀), hydrogenated farnesol (branched C₁₅), and hydrogenated phytol (branched C₂₀).

Non-limiting examples of phenyls substituted with a C₂-C₄₀ alkyl include octylphenyl, nonylphenyl, decylphenol, dodecylphenyl, hexadecylphenyl, octadecylphenyl, isooctylphenol, and sec-butylphenyl.

C₈-C₄₀ alicyclic groups may be, for example, groups derived from sterols of animal origin, such as cholesterol, lanosterol, and 7-dehydrocholesterol; or derivatives of plant origin, such as phytosterol, stigmasterol, or campesterol; or derivatives obtained from microorganisms, such as ergosterol or mycrosterol. Other useful C₈-C₄₀ alicyclics that may be used include, but are not limited to, cyclooctyl, cyclododecyl, adamantyl, and decahydronaphthyl, and groups derived from natural C₈-C₄₀ alicyclics compounds such as pinene, hydrogenated retinol, camphor, and isobornyl alcohol.

The C₂-C₄₀ alkyl groups substituted with an aryl group may be, for example, 2-phenylethyl, 2,4-diphenybutyl, 2,4,6-triphenylhexyl, 4-phenylbutyl, 2-methyl-2-phenylethyl, or 2,4,6-tris(1′-phenylethyl)phenyl.

Non-limiting examples of C₈-C₄₀ complex esters that may be used as end (iii)′ include hydrogenated castor oil (for example, 12-hydroxystearic acid triglyceride); 1,2-diacyl glycerols, such as 1,2-distearyl glycerol, 1,2-dipalmitoyl glycerol, and 1,2-dimyristoyl glycerol; di-, tri-, or polyesters of sugars, such as 3,4,6-tristearyl glucose or 2,3-dilauryl fructose; and sorbitan esters such as those described in U.S. Pat. No. 4,600,761.

The at least one associative vinyl monomer that may be used according to the present disclosure may be prepared via any method known in the prior art. Reference may be made, for example, to U.S. Pat. Nos. 4,421,902; 4,384,096; 4,514,552; 4,600,761; 4,616,074; 5,294,692; 5,292,843; 5,770,760 and 5,412,142.

In at least one embodiment, the at least one associative vinyl monomer that may be used according to the present disclosure is chosen from compounds of formula (III):

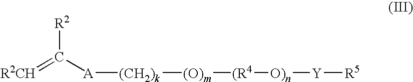

wherein:

each R² is independently chosen from a hydrogen atom, a methyl group, a —C(O)OH group, and a —C(O)OR³ group;

wherein R³ is a C₁-C₃₀ alkyl;

A is chosen from —CH₂C(O)O—, —C(O)O—, —O—, CH₂O, —NHC(O)NH—, —C(O)NH—, —Ar—(CE₂)_{z}-NHC(O)O—, —Ar—(CE₂)_{z}-NHC(O)NH—, and —CH₂CH₂—NHC(O)— groups;

Ar is a divalent aryl group;

E is chosen from a hydrogen atom and a methyl group;

z is an integer ranging from 0 to 1;

k is an integer ranging from 0 to 30;

m is an integer ranging from 0 to 1, with the provisos that when k is 0, m is 0, and when k is an integer ranging from 1 to 30, then m is 1;

(R⁴—O)ₙ is a polyoxyalkylene, which is a homopolymer, a random copolymer, or a block copolymer, comprising C₂-C₄ oxyalkylene units;

R⁴ is chosen from C₂H₄, C₃H₆, C₄H₈, and mixtures thereof;

n is an integer ranging from 5 to 250;

Y is chosen from —R⁴O—, —R⁴NH—, —C(O)—, —C(O)NH—, R⁴NHC(O)NH—, and —C(O)NHC(O)—;

R⁵ is chosen from substituted and unsubstituted linear C₈-C₄₀ alkyl groups, branched C₈-C₄₀ alkyl groups, C₈-C₄₀ alicyclic groups, phenyls substituted with a C₂-C₄₀ alkyl group, C₂-C₄₀ alkyl groups substituted with an aryl group, and C₈-C₈₀ complex esters, wherein

the alkyl group R⁵ optionally comprises at least one substituent chosen from hydroxyl, alkoxy and halo groups.

According to at least one embodiment, the at least one associative vinyl monomer is chosen from polyethoxylated cetyl (meth)acrylates, polyethoxylated cetearyl (meth)acrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-tris(1′-phenylethyl)phenyl (meth)acrylates, polyethoxylated hydrogenated castor oil (meth)acrylates, polyethoxylated canola (meth)acrylates, polyethoxylated cholesteryl (meth)acrylates, and mixtures thereof, wherein the polyethoxylated portion of the monomer comprises from 5 to 100 ethylene oxide units, such as from 10 to 80 ethylene oxide units and further such as from 15 to 60 ethylene oxide units.

In another embodiment, the at least one associative vinyl monomer is chosen from polyethoxylated cetyl methacrylates, polyethoxylated cetearyl methacrylates, polyethoxylated stearyl (meth)acrylates, polyethoxylated arachidyl (meth)acrylates, polyethoxylated behenyl (meth)acrylates, and polyethoxylated lauryl (meth)acrylates, wherein the polyethoxylated portion of the monomer comprises from 10 to 80 ethylene oxide units, such as from 15 to 60 ethylene oxide units and further such as from 20 to 40 ethylene oxide units.

In a further embodiment, the at least one associative vinyl monomer is present in an amount ranging from 0.001% to 25% by weight of the monomer mixture, for instance from 0.01% to 15% by weight of the monomer mixture, and further such as from 0.1% to 10% by weight, relative to the total weight of the monomer mixture.

The at least one semi-hydrophobic vinyl surfactant monomer optionally present in the monomer mixture can moderate the associative properties of the cationic associative polymers that comprise them, thus may produce aqueous gels having a very good texture and very good rheological properties.

As used herein, the term “semi-hydrophobic vinyl surfactant monomer” means a monomer with a structure similar to that of an associative monomer, but which has a substantially non-hydrophobic end and thus does not give the polymers associative properties.

The associative property of a polymer is linked to the property in a given medium, of the molecules of the said polymer to associate with each other, or to associate with molecules of a co-agent, such as a surfactant, which is reflected in a certain concentration range by an increase in the viscosity of the medium.

The at least one semi-hydrophobic vinyl surfactant monomer may be compounds comprising two parts:

A. an unsaturated end group to allow addition polymerization with the other monomers of the reaction mixture, and

B. a polyoxyalkylene group to attenuate the associations between the hydrophobic groups of the polymer or the hydrophobic groups of the other materials that may be present in the composition comprising the polymer.

The end A providing the vinyl or ethylenic unsaturation for the addition polymerization may be, for example, derived from an α,β-ethylenically unsaturated monocarboxylic or dicarboxylic acid or anhydride, such as a C₃-C₄ monocarboxylic or dicarboxylic acid, or an anhydride of this acid. Alternatively, the end A may be derived from an allylic ether, a vinyl ether, or a nonionic unsaturated urethane.

The polymerizable unsaturated end A may also be derived from a C₈-C₃₀ unsaturated fatty acid comprising at least one free carboxyl functional group. This C₈-C₃₀ group forms part of the unsaturated end A and is different from the pendent hydrophobic groups of the associative monomers, which are separated from the unsaturated end of the associative monomer by a hydrophilic spacer group.

The polyoxyalkylene portion B comprises a long-chain polyoxyalkylene segment, which is similar to the hydrophilic portion of the associative monomers. In at least one embodiment, the polyoxyalkylene portions B include C₂-C₄ polyoxyethylene, polyoxypropylene, and polyoxybutylene units comprising from 5 to 250 oxyalkylene units, such as from 10 to 100 oxyalkylene units. When the at least one semi-hydrophobic vinyl surfactant monomer comprises more than one type of oxyalkylene unit, these units may be distributed randomly, non-randomly, or in blocks.

According to at least one embodiment, the at least one semi-hydrophobic vinyl surfactant monomer is chosen from compounds of formula (IV) and (V):

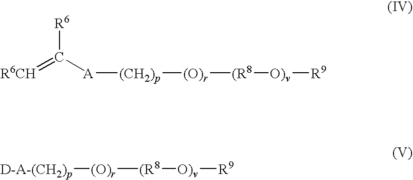

wherein:

each R⁶ independently is chosen from a hydrogen atom, C₁-C₃₀ alkyl, —C(O)OH, and C(O)OR⁷;

wherein R⁷ is a C₁-C₃₀ alkyl;

A is chosen from —CH₂C(O)O—, —C(O)O—, —O—, —CH₂O, —NHC(O)NH—, —C(O)NH—, —Ar—(CE₂)—NHC(O)O—, —Ar—(CE₂)_{z}-NHC(O)NH—, and —CH₂CH₂NHC(O)—;

Ar is a divalent aryl group;

E is chosen from a hydrogen atom and a methyl group;

z is an integer ranging from 0 to 1;

p is an integer ranging from 0 to 30;

r is an integer ranging from 0 to 1, with the provisos that when p is 0, then r is 0, and when p is an integer ranging from 1 to 30, then r is 1;

(R₈—O)ᵥ is a polyoxyalkylene which is a homopolymer, a random copolymer, or a block copolymer comprising C₂-C₄ oxyalkylene units, wherein R⁸ is chosen from C₂H₄, C₃H₆, C₄H₈, and mixtures thereof, and v is an integer ranging from 5 to 250;

R⁹ is chosen from a hydrogen atom and a C₁-C₄ alkyl; and

D is chosen from C₈-C₃₀ alkenyl groups optionally substituted with a carboxyl group.

In at least one embodiment, the monomer mixture comprises at least one semi-hydrophobic vinyl surfactant monomer chosen from one of the following formulae:

CH₂═CH—O(CH₂)ₐO(C₃H₆O)_{b}(C₂H₄O)_{c}H, and

CH₂═CHCH₂O(C₃H₆O)_{d}(C₂H₄O)₉H;

wherein:

a is an integer ranging from 2 to 4;

b is an integer ranging from 1 to 10;

c is an integer ranging from 5 to 50;

d is an integer ranging from 1 to 10; and

e is an integer ranging from 5 to 50.

Non-limiting examples of the at least one semi-hydrophobic vinyl surfactant monomer include polymerizable emulsifiers sold under the references EMULSOGEN® R109, R208, R307, RAL109, RAL208, and RAL307 by the company Clariant; BX-AA-E5P5 sold by the company Bimax; and MAXEMUL® 5010 and 5011 sold by the company Uniqema. In at least one embodiment, the at least one semi-hydrophobic vinyl surfactant monomer is chosen from EMULSOGEN® R208, R307, and RAL 307.

According to the manufacturers:

EMULSOGEN® P109 is a random ethoxylated/propoxylated 1,4-butanediol vinyl ether having the empirical formula:

CH₂═CH—O(CH₂)₄O(C₃H₆O)₄(C₂H₄O)₁₀H;

EMULSOGEN® R208 is a random ethoxylated/propoxylated 1,4-butanediol vinyl ether having the empirical formula:

CH₂═CH—O(CH₂)₄O(C₃H₆O)₄(C₂H₄O)₂₀H;

EMULSOGEN® R307 is a random ethoxylated/propoxylated 1,4-butanediol vinyl ether having the empirical formula:

CH₂═CH—O(CH₂)₄O(C₃H₆O)₄(C₂H₄O)₃₀H;

EMULSOGEN® RAL 109 is a random ethoxylated/propoxylated allylic ether having the empirical formula:

CH₂═CHCH₂—O(C₃H₆O)₄(C₂H₄O)₁₀H;

EMULSOGEN® RAL 208 is a random ethoxylated/propoxylated allylic ether having the empirical formula:

CH₂═CHCH₂—O(C₃H₆O)₄(C₂H₄O)₂₀H;

EMULSOGEN® RAL 307 is a random ethoxylated/propoxylated allylic ether having the empirical formula:

CH₂═CHCH₂—O(C₃H₆O)₄(C₂H₄O)₃₀H;

MAXEMUL® 5010 is a hydrophobic carboxylated C₁₂-C₁₅ alkenyl, ethoxylated with 24 ethylene oxide units;

MAXEMUL® 5011 is a hydrophobic carboxylated C₁₂-C₁₅ alkenyl, ethoxylated with 34 ethylene oxide units; and

BX-AA-E5P5 is a random ethoxylated/propoxylated allylic ether having the empirical formula:

CH₂═CHCH₂—O(C₃H₆O)₅(C₂H₄O)₅H.

The amount of the at least one semi-hydrophobic vinyl surfactant monomer used in the preparation of the at least one cationic polymer (i) used in the composition according to the present disclosure may vary widely and also depend on the final rheological properties desired for the polymer.

When present, the at least one semi-hydrophobic vinyl surfactant monomer may be present in an amount ranging from 0.01% to 25% by weight, for example from 0.1% to 10% by weight, relative to the total weight of the monomer mixture.

The at least one cationic polymer (i) used in the composition, according to the present disclosure, is prepared from a monomer mixture that may comprise at least one hydroxylated nonionic vinyl monomer, which are ethylenically unsaturated monomers comprising at least one hydroxyl substituent.

With respect to the at least one hydroxylated nonionic vinyl monomers, non-limiting mention may be made of hydroxylated C₁-C₆ alkyl (meth)acrylates, such as hydroxylated C₁-C₄ alkyl (meth)acrylates, for instance 2-hydroxyethyl methacrylate (HEMA), 2-hydroxyethyl acrylate (2-HEA), and 3-hydroxypropyl acrylate; hydroxylated C₁-C₄ alkyl(meth)acrylamides, such as N-(2-hydroxyethyl)methacrylamide, N-(2-hydroxyethyl)acrylamide, N-(3-hydroxypropyl)acrylamide, and N-(2,3-dihydroxypropyl)acrylamide; and mixtures thereof. Non-limiting mention may also be made of allyl alcohol, glyceryl monoallyl ether, 3-methyl-3-buten-1-ol, and vinyl alcohol precursors, and equivalents thereof, such as vinyl acetate.

The at least one hydroxylated nonionic vinyl monomer may be present in an amount ranging from 0% to 10% by weight, relative to the total weight of the monomer mixture. In at least one embodiment, the at least one hydroxylated nonionic vinyl monomer is present in an amount ranging from 0.01% to 10% by weight, for example from 1% to 8% and further such as from 1% to 5% by weight, relative to the total weight of the monomer mixture.

The at least one cationic polymer (i) in the composition according to the present disclosure is prepared from a monomer mixture that may comprise at least one crosslinking monomer for introducing branches and controlling the molecular mass.

Polyunsaturated crosslinking agents that may be used are well known in the prior art. Monounsaturated compounds with a reactive group capable of crosslinking a copolymer formed before, during or after the polymerization may be used. Other crosslinking monomers that may be used include, but are not limited to, polyfunctional monomers comprising multiple reactive groups such as peroxide and isocyanate groups and hydrolysable silane groups. Many polyunsaturated compounds may be used to generate a partially or substantially crosslinked three-dimensional network.

Non-limiting examples of polyunsaturated crosslinking monomers that may be used include polyunsaturated aromatic monomers, such as divinylbenzene, divinylnaphthalene, and trivinylbenzene; polyunsaturated alicyclic monomers such as 1,2,4-trivinylcyclohexane; difunctional phthalic acid esters such as diallyl phthalate; polyunsaturated aliphatic monomers such as dienes, trienes, and tetraenes, for example, isoprene, butadiene, 1,5-hexadiene, 1,5,9-decatriene, 1,9-decadiene, and 1,5 heptadiene.

Other non-limiting examples of polyunsaturated crosslinking monomers that may be used include polyalkenyl ethers, such as triallylpentaerythritol, diallylpentaerythritol, diallylsucrose, octaallylsucrose, and trimethylolpropane diallyl ether; polyunsaturated esters of polyalcohols or of polyacids, such as 1,6-hexanediol di(meth)acrylate, tetramethylene tri(meth)acrylate, allyl acrylate, diallyl itaconate, diallyl fumarate, diallyl maleate, trimethylolpropane tri(meth)acrylate, trimethylolpropane di(meth)acrylate, and polyethylene glycol di(meth)acrylate; alkylenebisacrylamides, such as methylenebisacrylamide and propylenebisacrylamide; hydroxylated and carboxylated derivatives of methylenebisacrylamide, such as N,N′-bismethylol methylenebisacrylamide; polyethylene glycol di(meth)acrylates, such as ethylene glycol di(meth)acrylate, diethylene glycol di(meth)acrylate, and triethylene glycol di(meth)acrylate; polyunsaturated silanes, such as dimethyldivinylsilane, methyltrivinylsilane, allyldimethylvinylsilane, diallyldimethylsilane, and tetravinylsilane; and polyunsaturated stannanes, such as tetraallyltin and diallyldimethyltin.

Non-limiting examples of monounsaturated crosslinking monomers that may be used and that bear a reactive group may be N-methylolacrylamides; N-alkoxy(meth)acrylamides, wherein the alkoxy group is a C₁-C₁₈ group; and unsaturated hydrolysable silanes, such as triethoxyvinylsilane, tris-isopropoxyvinylsilane, and 3-triethoxysilylpropyl methacrylate.

Polyfunctional crosslinking monomers that may be used and that comprise several reactive groups include, for example, hydrolysable silanes, such as ethyltriethoxysilane and ethyltrimethoxysilane; epoxidized hydrolysable silanes, such as 2-(3,4-epoxycyclohexyl)ethyltriethoxysilane and 3-glycidoxypropyltrimethoxysilane; polyisocyanates, such as 1,4-diisocyanatobutane, 1,6-diisocyanatohexane, 1,4-phenylenediisocyanate, and 4,4′-oxybis(phenylisocyanate); unsaturated epoxides, such as glycidyl methacrylate and allyl glycidyl ether; and polyepoxides, such as diglycidyl ether, 1,2,5,6-diepoxyhexane, and ethylene glycol diglycidyl ether.

Further examples of polyunsaturated crosslinking monomers that may be used include, but are not limited to, ethoxylated polyols, such as diols, triols, and bis-phenols, ethoxylated with from 2 to 100 mol of ethylene oxide per mole of hydroxyl functional group and ending with a polymerizable unsaturated group such as a vinyl ether, an allyl ether, an acrylate ester, or a methacrylate ester. Such crosslinking monomers may be, for example, ethoxylated bisphenol A dimethacrylate, ethoxylated bisphenol F dimethacrylate, and ethoxylated trimethylolpropane trimethacrylate.

Other non-limiting examples of ethoxylated crosslinking monomers that may be used include the crosslinking agents derived from ethoxylated polyols, described in U.S. Pat. No. 6,140,435.

Further non-limiting examples of crosslinking monomers are acrylate and methacrylate esters of polyols comprising at least two acrylate or methacrylate ester groups, such as trimethylolpropane triacrylate (TMPTA), trimethylolpropane dimethacrylate, triethylene glycol dimethacrylate (TEGDMA), and ethoxylated (30) bisphenol A dimethacrylate (EOBDMA).

The at least one crosslinking monomer may be present in an amount ranging from 0% to 5% by weight, relative to the weight of the monomer mixture. According to one embodiment, the at least one crosslinking monomer is present in an amount ranging from 0.001% to 5% by weight, for example from 0.05% to 2% by weight and further, for example, from 0.1% to 1% by weight, relative to the total weight of the monomer mixture.

The monomer mixture may comprise at least one chain-transfer agent.

Non-limiting mention may be made of chain-transfer agents, for example, thiol compounds, disulfide compounds, such as C₁-C₁₈ mercaptans, mercaptocarboxylic acids, mercaptocarboxylic acid esters, thioesters, C₁-C₁₈ alkyl disulfides, aryl disulfides, and polyfunctional thiols; phosphites and hypophosphites; haloalkyl compounds, such as carbon tetrachloride and bromotrichloromethane; and unsaturated chain-transfer agents, such as α-methylstyrene.

The polyfunctional thiols are, for example, trifunctional thiols, such as trimethylolpropane tris(3-mercaptopropionate), tetrafunctional thiols, such as pentaerythritol tetrakis(thioglycolate) and pentaerythritol tetrakis(thiolactate); hexafunctional thiols, such as pentaerythritol hexakis(thioglycolate).

In another embodiment, the at least one chain-transfer agent may be at least one catalytic chain-transfer agent that reduces the molecular weight of the addition polymers during the free-radical polymerization of the vinyl monomers. Examples that may be mentioned include, but are not limited to, cobalt complexes, such as cobalt (II) chelates. The at least one catalytic chain-transfer agent may also be used at low concentrations relative to the thiolated chain-transfer agents.

Non-limiting examples of the at least one chain-transfer agent that may be mentioned include octyl mercaptan, n-dodecyl mercaptan, t-dodecyl mercaptan, hexadecyl mercaptan, octadecyl mercaptan (ODM), isooctyl 3-mercaptopropionate (IMP), butyl 3-mercaptopropionate, 3-mercaptopropionic acid, butyl thioglycolate, isooctyl thioglycolate, and dodecyl thioglycolate.

The at least one chain-transfer agent may be present in an amount ranging from 0% to 10% by weight, relative to the total weight of the monomer mixture. In at least one embodiment, the at least one chain-transfer agent is present in an amount ranging from 0.1% to 5% by weight, relative to the total weight of monomers.

The monomer mixture for preparing the at least one cationic polymer (i) used in the composition according to the present disclosure may comprise at least one polymeric stabilizer for obtaining stable dispersions or emulsions. In at least one embodiment, the at least one polymer is water-soluble. Non-limiting examples include synthetic polymers, such as polyvinyl alcohols, partially hydrolysed polyvinyl acetates, polyvinylpyrrolidone, polyacrylamides, polymethacrylamides, carboxylated addition polymers, and polyalkyl vinyl ethers; water-soluble natural polymers, such as gelatin, pectins, alginates, and casein; and modified natural polymers, such as methylcellulose, hydroxypropylcellulose, carboxymethylcellulose, and allylic hydroxyethylcelluloses.

The at least one polymeric stabilizer may be present in an amount ranging from 0% to 2% by weight, relative to the total weight of the monomer mixture, for instance in an amount ranging from 0.0001% to 1% by weight and further such as from 0.01% to 0.5% by weight, relative to the weight of the monomer mixture.

According to one embodiment, the monomer mixture comprises, relative to the total weight of the monomer mixture:

a) from 10% to 70% by weight of at least one vinyl monomer substituted with at least one amino group,

b) from 20% to 80% by weight of at least one hydrophobic nonionic vinyl monomer,

c) from 0.001% to 25% by weight of at least one associative vinyl monomer,

d) from 0 to 25% by weight of at least one semi-hydrophobic vinyl surfactant monomer,

e) from 0 to 10% by weight of at least one hydroxylated nonionic vinyl monomer,

f) from 0 to 5% by weight of at least one crosslinking monomer,

g) from 0 to 10% by weight of at least one chain-transfer agent, and

h) from 0 to 2% by weight of at least one polymeric stabilizer.

In yet another embodiment, the monomer mixture comprises, relative to the total weight of the monomer mixture:

a) from 20% to 60% by weight of at least one vinyl monomer substituted with at least one amino group,

b) from 20% to 70% by weight of at least one hydrophobic nonionic vinyl monomer,

c) from 0.01% to 15% by weight of at least one associative vinyl monomer,

d) from 0.1% to 10% by weight of at least one semi-hydrophobic vinyl surfactant monomer,

e) from 0.01% to 10% by weight of at least one hydroxylated nonionic vinyl monomer,

f) from 0.001% to 5% by weight of at least one crosslinking monomer,

g) from 0.001% to 10% by weight of at least one chain-transfer agent, and

h) from 0 to 2% by weight of at least one polymeric stabilizer.

According to at least one embodiment of the present disclosure, the monomer mixture for preparing the at least one cationic polymer (i) used in the composition disclosed herein comprises, relative to the total weight of the monomer mixture:

a) from 20% to 50% by weight of at least one vinyl monomer substituted with at least one amino group chosen from:
3-(N,N-dimethylamino)propyl (meth)acrylate,
N′-(3-N,N-dimethylamino)propyl(meth)acrylamide,
2-(N,N-dimethylamino)ethyl (meth)acrylate,
2-(N,N-diethylamino)ethyl (meth)acrylate,
2-(tert-butylamino)ethyl (meth)acrylate,
2-(N,N-dimethylamino)propyl(meth)acrylamide, and
2-(N,N-dimethylamino)neopentyl acrylate,

b) from 50% to 65% by weight of at least one hydrophobic nonionic vinyl monomer chosen from C₁-C₃₀ alkyl esters of acrylic acid, C₁-C₃₀ alkyl esters of methacrylic acid, and mixtures thereof,

c) from 0.1% to 10% by weight of at least one associative vinyl monomer chosen from polyethoxylated cetyl methacrylates, polyethoxylated cetearyl methacrylates, polyethoxylated stearyl (meth)acrylates, polyethoxylated arachidyl (meth)acrylates, polyethoxylated behenyl (methacrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-tris(1′-phenylethyl)phenyl (methacrylates, polyethoxylated hydrogenated castor oil (meth)-acrylates, polyethoxylated canola (meth)acrylates, and polyethoxylated cholesteryl (meth)acrylates, and mixtures thereof,

d) from 0.1% to 10% by weight of at least one semi-hydrophobic vinyl surfactant monomer chosen from one of the following formulae:

CH₂═CH—O(CH₂)ₐO(C₃H₆O)_{b}(C₂H₄O)_{c}H, and

CH₂═CHCH₂O(C₃H₆O)_{d}(C₂H₄O)ₑH;

wherein:

a is an integer ranging from 2 to 4;

b is an integer ranging from 1 to 10;

c is an integer ranging from 5 to 50;

d is an integer ranging from 1 to 10; and

e is an integer ranging from 5 to 50;

e) from 0% to 10% by weight of at least one hydroxylated nonionic vinyl monomer,

f) from 0% to 5% by weight of at least one crosslinking monomer,

g) from 0% to 10% by weight of at least one chain-transfer agent, and

h) from 0% to 2% by weight of at least one polymeric stabilizer.

In at least one embodiment, the at least one cationic polymer (i) disclosed herein is chosen from polymers derived from the polymerization of the following monomer mixture:

a di(C₁-C₄ alkyl)amino(C₁-C₆ alkyl)methacrylate,

at least one C₁-C₃₀ alkyl ester of (meth)acrylic acid,

a C₁₀-C₃₀ alkyl methacrylate polyethoxylated comprising from 20 to 30 mol of ethylene oxide,

a 30/5 polyethylene glycol/polypropylene glycol allyl ether,

a hydroxy(C₂-C₆ alkyl)methacrylate, and

an ethylene glycol dimethacrylate.

Among the cationic polymers (i) used in the composition according to the present disclosure, non-limiting mention may be made, for example, of the compound sold by the company Noveon under the name CARBOPOL® Aqua CC Polymer and which corresponds to the INCI name Polyacrylate-1 Crosspolymer.

Polyacrylate-1 Crosspolymer is the product of polymerization of a monomer mixture comprising:

a di(C₁-C₄ alkyl)amino(C₁-C₆ alkyl)methacrylate,

at least one C₁-C₃₀ alkyl ester of (meth)acrylic acid,

a polyethoxylated C₁₀-C₃₀ alkyl methacrylate (20-25 mol of ethylene oxide units),

a 30/5 polyethylene glycol/polypropylene glycol allyl ether,

a hydroxy(C₂-C₆ alkyl)methacrylate, and

an ethylene glycol dimethacrylate.

The at least one cationic polymer (i) used in the compositions according to the present disclosure may be present in an amount ranging from 0.01% to 10% by weight, for example from 0.05% to 5% by weight and further for example from 0.1% to 1% by weight, relative to the total weight of the composition.

As disclosed herein, the at least one cationic polymer (i) used in the composition may be prepared via conventional polymerization techniques, such as emulsion polymerization. The polymerization may be performed via a simple batch process, or via a controlled addition process, or the reaction may be initiated in a small reactor and the mass of monomers may then be added in a controlled manner to the reactor (seeding process). For example, the polymerization may be performed at a reaction temperature ranging from 20 to 80° C., although higher or lower temperatures may be used. To facilitate the emulsification of the monomer mixture, the emulsion polymerization is performed in the presence of a surfactant that is present in an amount ranging from 1% to 10% by weight, for instance from 3% to 8% by weight, and further such as from 5% to 7% by weight, relative to the total weight of the emulsion. The emulsion polymerization reaction medium also comprises at least one radical initiator, which may be present, for example, in an amount ranging from 0.01% to 3% by weight, relative to the total weight of the monomer mixture. The polymerization may be performed in an aqueous or aqueous-alcoholic medium at a neutral or weakly alkaline pH.

In a typical polymerization, the monomer mixture is added with stirring to a solution of emulsifying surfactants, such as a nonionic surfactant, for example, a linear or branched alcohol ethoxylate, or a mixture of nonionic and anionic surfactants, such as fatty alkyl sulfates or alkyl sulfonates of fatty alcohols, in a suitable amount of water, in a suitable reactor, to prepare the monomer emulsion. The emulsion is deoxygenated via any known method, and the polymerization reaction is then initiated by adding a polymerization catalyst (initiator) such as sodium persulfate, or any other suitable addition polymerization catalyst, as is well known in the field of polymers. The reaction is stirred until the polymerization is complete, for example, for a time ranging from 4 hours to 16 hours. The monomer emulsion may be heated to a temperature ranging from 20 to 80° C. before adding the initiator, if so desired. The amount of unreacted monomers may be removed by adding an additional amount of catalyst. The polymer emulsion obtained may be discharged from the reactor and packaged for storage or used. Optionally, the pH or other physical or chemical characteristics of the emulsion may be adjusted before discharging the emulsion from the reactor. The emulsion produced can have a total solids content ranging between 10% and 40% by weight. The total amount of polymers in the emulsion obtained can range between 15% and 35% by weight, and, for example, not more than 25% by weight.

Surfactants that are suitable for facilitating the emulsion polymerization include, but are not limited to surfacts conventionally used in emulsion polymerizations, such as nonionic, anionic, amphoteric, and cationic surfactants, and mixtures thereof. In at least one embodiment, nonionic surfactants, anionic surfactants, and mixtures thereof are used.

The polymerization may be performed in the presence of at least one free-radical initiator. These initiators may be chosen from insoluble inorganic persulfate compounds, such as ammonium persulfate, potassium persulfate, and sodium persulfate; peroxides, such as hydrogen peroxide, benzoyl peroxide, acetyl peroxide, and lauryl peroxide; organic hydroperoxides, such as cumene hydroperoxide and t-butyl hydroperoxide; organic peracids, such as peracetic acid; and oil-soluble free-radical generators, such as 2,2′-azobisisobutyronitrile, and mixtures thereof. The peroxides and peracids may be optionally activated with reducing agents, such as sodium bisulfite or ascorbic acid, transition metals, or hydrazine. Suitable free-radical initiators include, but are not limited to, water-soluble azo polymerization initiators such as 2,2′-azobis(tert-alkyl) compounds bearing a water-solubilizing substituent on the alkyl group. Non-limiting examples of azo polymerization catalysts that may be used include the VAZO® free-radical initiators sold by the company DuPont, such as VAZO® 44 (2,2′-azobis(2-4,5-dihydroimidazolyl)propane), VAZO® 56 (2,2′-azobis(2-methylpropionamidine) dihydrochloride) and VAZO® 68 (4,4′-azobis(4-cyanovaleric acid)).

The silicones that may be used in accordance with the present disclosure are, for example, insoluble in the composition and may be in the form of oils, waxes, resins, or gums.

As used herein, “silicone” is understood to mean any organosilicon polymer or oligomer of linear or cyclic, branched or crosslinked structure, of variable molecular weight, obtained by polymerization and/or polycondensation of suitably functionalized silanes, and consisting essentially of a repetition of main units wherein the silicon atoms are linked together via oxygen atoms (siloxane bond —Si—O—Si—), optionally substituted hydrocarbon-based radicals being directly linked via a carbon atom to the said silicon atoms. The hydrocarbon-based radicals that are the most common are alkyl radicals, for instance, C₁-C₁₀ alkyl radicals, for example methyl radicals, fluoroalkyl radicals, and aryl radicals, such as phenyl.

As disclosed herein, “amino silicone” means any silicone comprising at least one primary, secondary or tertiary amine function or at least one quaternary ammonium group.

Non-limiting examples of the at least one amino silicone used in the cosmetic composition according to the present disclosure include:

(a) the compounds of formula (VI):

(R¹)ₐ(T)₃₋ₐ-Si[OSi(T)₂]ₙ-[OSi(T)_{b}(R¹)_{2-b}]ₘ—OSi(T)₃₋ₐ-(R¹)ₐ (VI)

wherein:

T is chosen from a hydrogen atom, a phenyl, a hydroxyl (—OH), a C₁-C₈ alkyl radical, such as a methyl group, and a C₁-C₈ alkoxy radical, for example a methoxy group,

a is an integer ranging from 0 to 3,

b is an integer ranging from 0 to 1,

m and n are integers such that the sum (n+m) can range, for example, from 1 to 2000, such as from 50 to 150, wherein n, for example, is an integer ranging from 0 to 1999, such as from 49 to 149, and m, for instance, is an integer ranging from 1 to 2000, for example from 1 to 10,

R¹ is a monovalent radical of formula —C_{q}H_{2q}L wherein q is an integer ranging from 2 to 8 and L is an optionally quaternized amino group chosen from:

—N(R²)—CH₂—CH₂—N(R²)₂;

—N(R²)₂;

—N⁺(R²)₃Q⁻;

N⁺(R²)(H)₂Q⁻;

—N⁺(R²)₂HQ⁻; and

—N(R²)—CH₂—CH₂—N⁺(R²)(H)₂Q⁻,

wherein R² is chosen from a hydrogen atom, a phenyl, a benzyl and a saturated monovalent hydrocarbon-based radical, for example a C₁-C₂₀ alkyl radical, and Q⁻ is a halide ion chosen from fluoride, chloride, bromide, and iodide.

In at least one embodiment, the at least one amino silicone corresponding to the definition of formula (VI) are chosen from compounds of formula (VII):

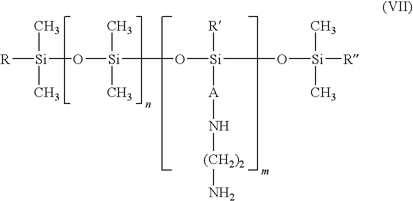

wherein R, R′, and R″, which may be identical or different, are chosen from C₁-C₄ alkyl radicals, such as CH₃, C₁-C₄ alkoxy radicals, for instance methoxy groups, and OH; A is chosen from a linear and branched C₃-C₈ alkylene radical, such as a C₃-C₆ alkylene radical; and m and n are integers dependent on the molecular weight and whose sum ranges from 1 and 2000.

According to at least one embodiment, R, R′, and R″, which may be identical or different, are chosen from a C₁-C₄ alkyl radical and a hydroxyl radical, A is a C₃ alkylene radical and m and n are such that the weight-average molecular mass of the compound may range from approximately 5000 to 500 000. Compounds of this type are referred to in the CTFA dictionary as “amodimethicones.”

In another embodiment, R, R′, and R″, which may be identical or different, are chosen from a C₁-C₄ alkoxy radical and a hydroxyl radical, wherein at least one of the radicals R or R″ is an alkoxy radical and A is a C₃ alkylene radical. The hydroxyl/alkoxy molar ratio is, for example, between 0.2/1 and 0.4/1 and, in at least one embodiment, equal to 0.3/1. Moreover, m and n are such that the weight-average molecular mass of the compound may range between 2000 and 10⁶. According to at least one embodiment, n is an integer between 0 and 999 and m is an integer between 1 and 1000, the sum of n and m being between 1 and 1000.

In this category of compounds, non-limiting mention may be made of the product BELSIL® ADM 652 sold by Wacker.

According to another embodiment, R and R″, which are different, are chosen from a C₁-C₄ alkoxy radical and a hydroxyl radical, wherein at least one of the radicals R or R″ is an alkoxy radical, R′ is a methyl radical, and A is a C₃ alkylene radical. The hydroxyl/alkoxy molar ratio is, for instance, between 1/0.8 and 1/1.1, and is, in at least one embodiment, equal to 1/0.95. Moreover, m and n are such that the weight-average molecular mass of the compound may be between 2000 and 200 000. In at least one embodiment, n is an integer between 0 and 999 and m is an integer between 1 and 1000, the sum of n and m being between 1 and 1000.

Non-limiting mention may be made of the product FLUID WR® 1300 sold by the company Wacker.

According to yet another embodiment, R and R″ are hydroxyl radicals, R′ is a methyl radical, and A is a C₄-C₈ radical, such as a C₄ alkylene radical. Moreover, m and n are such that the weight-average molecular mass of the compound is between 2000 and 10⁶. In one embodiment, n is an integer between 0 and 1999 and m is an integer between 1 and 2000, the sum of n and m being between 1 and 2000.

A product of this type is sold, for example, under the name DC 28299 by Dow Corning.

It should be noted that the molecular mass of these silicones is determined by gel permeation chromatography (ambient temperature, polystyrene standard; μ styragem columns; eluent THF; flow rate 1 mm/m; 200 μl of a solution containing 0.5% by weight of silicone are injected into THF and detection is performed by UV refractometry).

A product corresponding to the definition of formula (VI) is, for instance, the polymer known in the CTFA dictionary as “trimethylsilyl amodimethicone,” corresponding to formula (VIII):

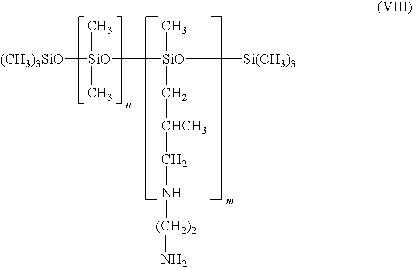

wherein n and m have the meanings given above in accordance with formula (VI).

Such compounds are described, for example, in European Patent Application No. EP 0 095 238; a compound of formula (VIII) is sold, for example, under the name Q2-8220 by the company OSI.

(b) the compounds of formula (IX):

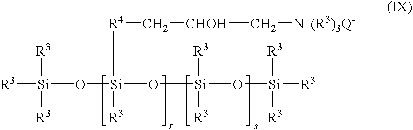

wherein:

R³ is chosen from a monovalent C₁-C₁₈ hydrocarbon-based radical, for example a C₁-C₁₈ alkyl radical and a C₂-C₁₈ alkenyl radical, such as a methyl group;

R⁴ is chosen from a divalent hydrocarbon-based radical, such as a C₁-C₁₈ alkylene radical and a divalent C₁-C₁₈ radical, for example a C₁-C₈ alkyleneoxy radical;

Q⁻ is a halide ion, for instance chloride;

r is an average statistical value ranging from 2 to 20, such as ranging from 2 to 8;

s is an average statistical value ranging from 20 to 200, for example ranging from 20 to 50.

Such compounds are described, for instance, in U.S. Pat. No. 4,185,087.

A non-limiting example of a compound falling within this class is the product sold by the company Union Carbide under the name UCAR SILICONE ALE 56.

(c) the quaternary ammonium silicones of formula (X):

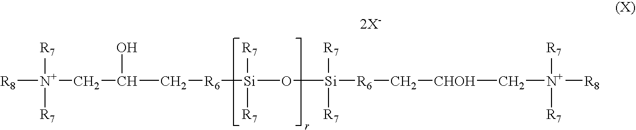

wherein:

R₇, which may be identical or different, is chosen from a monovalent C₁-C₁₈ hydrocarbon-based radical, such as a C₁-C₁₈ alkyl radical, a C₂-C₁₈ alkenyl radical, and a ring comprising 5 or 6 carbon atoms, for example a methyl group;

R₆ is chosen from a divalent hydrocarbon-based radical, for example a C₁-C₁₈ alkylene radical and a divalent C₁-C₁₈ radical, for example a C₁-C₈ alkyleneoxy radical linked to the Si via a SiC bond;

R₈, which may be identical or different, is chosen from a hydrogen atom and a monovalent C₁-C₁₈ hydrocarbon-based radical, for example a C₁-C₁₈ alkyl radical, a C₂-C₁₈ alkenyl radical, and a —R₆—NHCOR₇ radical;

X⁻ is an anion, for example a halide ion, such as chloride, or an organic acid salt (for example, acetate);

r is a mean statistical value ranging from 2 to 200, such as ranging from 5 to 100.

These silicones are described, for example, in European Patent Application No. EP 0 530 974.

(d) the amino silicones of formula (XI):

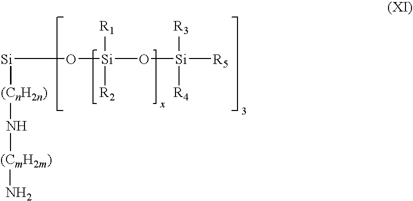

wherein:
R₁, R₂, R₃, and R₄, which may be identical or different, are chosen from C₁-C₄ alkyl radicals and phenyl groups,
R₅ is chosen from C₁-C₄ alkyl radical and a hydroxyl group,
n is an integer ranging from 1 to 5,
m is an integer ranging from 1 to 5, and
x is an integer ranging from 2 to 500, such as from 5 to 100.

Non-limiting examples of silicones that may be used in accordance with the present disclosure are polysiloxanes comprising amine groups such as amodimethicones and trimethylsilylamodimethicones (CTFA, 4th edition, 1997), for example silicones comprising quaternary ammonium groups.

In at least one embodiment of the present disclosure, when these compounds are used, they are combined with cationic and/or nonionic surfactants.

These compounds include, by way of non-limiting example, the product sold under the name CATIONIC EMULSION DC 929 by the company Dow Corning, which comprises, besides amodimethicone, a cationic surfactant comprising a mixture of products of formula:

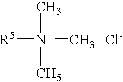

wherein R⁵ is chosen from a C₁₄-C₂₂ alkenyl radical and an alkyl radical derived from tallow fatty acids, and known, for example, under the CTFA name tallowtrimonium chloride, in combination with a nonionic surfactant of formula:

C₉H₁₉—C₆H₄—(OC₂H₄)₁₀—OH, known, for instance, under the CTFA name as Nonoxynol 10.

Non-limiting mention may also be made, for example, of the product sold under the name CATIONIC EMULSION DC 939 by the company Dow Corning, which comprises, amodimethicone, a cationic surfactant which is trimethylcetylammonium chloride, and a nonionic surfactant of formula: C₁₃H₂₇—(OC₂H₄)₁₂—OH, known, for example, under the CTFA name trideceth-12.

Another commercial product that may be used according to the present disclosure is the product sold, for instance, under the name DOW CORNING Q2 7224 by the company Dow Corning, comprising, in combination, the trimethylsilyl amodimethicone of formula (C) described above, a nonionic surfactant of formula: C₈H₁₇—C₆H₄—(OCH₂CH₂)₄₀—OH, known under the CTFA name octoxynol-40, a second nonionic surfactant of formula: C₁₂H₂₅—(OCH₂—CH₂)₆—OH, known under the CTFA name isolaureth-6, and propylene glycol.

According to the present disclosure, all the silicones may also be used in the form of emulsions or microemulsions.

According to the present disclosure, the at least one amino silicone may be present in an amount ranging from 0.001% to 20% by weight, for example from 0.01% to 10% by weight and further for example, from 0.1% to 3% by weight, relative to the total weight of the final composition.

The cosmetic composition according to the present disclosure comprises at least one cationic polymer (iii) whose cationic charge density is greater than 4 milliequivalents per gram (meq./g), for example, greater than or equal to 5 milliequivalents per gram (meq./g), and in at least one embodiment, ranging from 5 to 20 meq./g, for example, ranging from 5.5 to 10 meq./g.

The cationic charge density of a polymer corresponds to the number of moles of cationic charges per unit of mass of polymer under conditions in which this polymer is totally ionized. It may be determined by calculation if the structure of the polymer is known, i.e., the structure of the monomers constituting the polymer and their molar or weight proportion. It may also be determined experimentally via the Kjeldahl method, generally at pH of about 7 at room temperature.

The at least one cationic polymer with a cationic charge density of greater than or equal to 4 meq./g that may be used in accordance with the present disclosure may be chosen from all those already known for improving the cosmetic properties of the hair treated with compositions, for example, those described in European Patent Application No. EP 0 337 354 and in French Patent Application Nos. 2 270 846, 2 383 660, 2 598 611, 2470596, and 2519863.

As used herein, “cationic polymer” is understood to mean any polymer comprising cationic groups and/or groups that may be ionized into cationic groups.

The at least one cationic polymer is chosen from those containing units comprising primary, secondary, tertiary, and/or quaternary amine groups that either may form part of the main polymer chain or may be borne by a side substituent directly attached thereto.

The at least one cationic polymer used may have, for example, a number-average molecular mass of between approximately 500 and 5×10⁶, for example, between approximately 10³ and 3×10⁶. Among the at least one cationic polymer that may be mentioned include, but are not limited to polymers of the polyamine, polyamino amide, and polyquaternary ammonium type.

Among these polymers, non-limiting mention may be made of:

(1) homopolymers or copolymers derived from acrylic or methacrylic esters or amides and comprising at least one of the units of the following formulae:

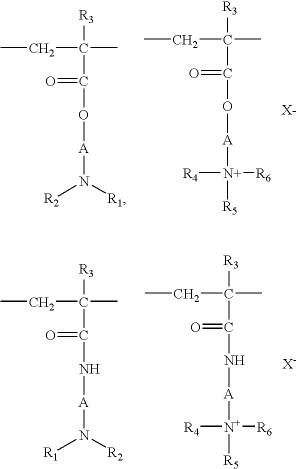

wherein:

R₃, which may be identical or different, is chosen from a hydrogen atom and a CH₃ radical;

A, which may be identical or different, is chosen from a linear and branched C₁-C₆ alkyl group, such as a C₂-C₃ alkyl group, and a hydroxyalkyl group comprising 1 to 4 carbon atoms;

R₄, R₅, and R₆, which may be identical or different, are chosen from C₁-C₁₈ alkyl groups and benzyl radicals, such as C₁-C₆ alkyl groups;

R₁ and R₂, which may be identical or different, are chosen from hydrogen atoms and C₁-C₆ alkyl groups, such as methyl or ethyl;

X⁻ is an anion derived from a mineral or organic acid, such as a methosulfate anion or a halide such as chloride or bromide.

The copolymers of family (1) can also comprise at least one unit derived from comonomers chosen from, by way of non-limiting example, acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Among these copolymers of family (1), non-limiting mention may be made of:

copolymers of acrylamide and of dimethylaminoethyl methacrylate quaternized with dimethyl sulfate or with a dimethyl halide,

the copolymers of acrylamide and of methacryloyloxyethyltrimethylammonium chloride described, for example, in European Patent Application No. EP 0 080 976,

the copolymers of acrylamide and of methacryloyloxyethyltrimethylammonium methosulfate,

quaternized or non-quaternized vinylpyrrolidone/dialkylaminoalkyl acrylate or methacrylate copolymers. These polymers are described in detail in French Patent Nos. 2 077 143 and 2 393 573,

dimethylaminoethyl methacrylate/vinylcaprolactam/vinylpyrrolidone terpolymers,

vinylpyrrolidone/methacrylamidopropyldimethylamine copolymers,

quaternized vinylpyrrolidone/dimethylaminopropylmethacrylamide copolymers, and

crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising olefinic unsaturation, such as methylene bisacrylamide. A crosslinked acrylamide/methacryloyloxyethyltrimethylammonium chloride copolymer (20/80 by weight) in the form of a dispersion comprising 50% by weight of the said copolymer in mineral oil may also be used. This dispersion is sold, for instance, under the name SALCARE® SC 92 by the company Ciba. A crosslinked methacryloyloxy-ethyltrimethylammonium chloride homopolymer containing about 50% by weight of the homopolymer in mineral oil or in a liquid ester can also be used. These dispersions are sold, for example, under the names SALCARE® SC 95 and SALCARE® SC 96 by the company Ciba.

(2) cyclopolymers of alkyldiallylamine or of dialkyldiallylammonium, such as the homopolymers or copolymers comprising, as main constituent of the chain, units of formula (XII) or (XIII):

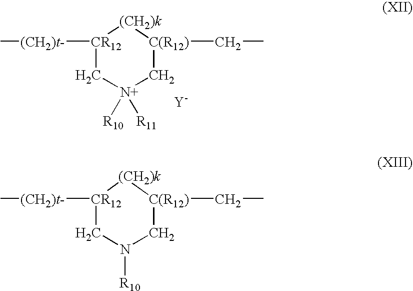

wherein k and t are integers ranging from 0 to 1, the sum k+t being 1; R₁₂ is chosen from a hydrogen atom and a methyl radical; R₁₀ and R₁₁, independently of each other, are chosen from C₁-C₆ alkyl groups, hydroxyalkyl groups wherein, for example, the alkyl group comprises 1 to 5 carbon atoms, and lower (C₁-C₄) amidoalkyl groups, or R₁₀ and R₁₁, together with the nitrogen atom to which they are attached, form heterocyclic groups such as piperidyl or morpholinyl; Y— is an anion, such as bromide, chloride, acetate, borate, citrate, tartrate, bisulfate, bisulfite, sulfate, and phosphate. These polymers are described, for example, in French Patent No. 2 080 759 and in its Certificate of Addition 2 190 406.

R₁₀ and R₁₁, independently of each other, are chosen from C₁-C₄ alkyl groups.

Among the polymers defined above, non-limiting mention may be made of the dimethyldiallylammonium chloride homopolymers sold, for instance, under the name MERQUAT 100 by the company Nalco (and its homologues of low weight-average molar mass) and copolymers of diallyldimethylammonium chloride and of acrylamide.

(3) quaternary copolymers of vinyllactam (vinylpyrrolidone and/or vinylcaprolactam) and of vinylimidazole.

(4) the quaternary diammonium polymer comprising repeating units of formula (XIV):

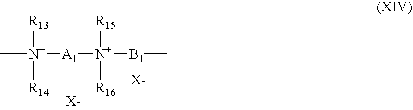

wherein:

R₁₃, R₁₄, R₁₅, and R₁₆, which may be identical or different, are chosen from C₁-C₂₀ aliphatic, alicyclic, and arylaliphatic radicals and lower C₁-C₆ hydroxyalkylaliphatic radicals, or R₁₃, R₁₄, R₁₅, and R₁₆, together or separately, constitute, with the nitrogen atoms to which they are attached, heterocycles optionally comprising a second heteroatom other than nitrogen, or R₁₃, R₁₄, R₁₅, and R₁₆ are chosen from linear and branched C₁-C₆ alkyl radicals substituted with a nitrile, ester, acyl, or amide group or a —CO—O—R₁₇-D or —CO—NH—R₁₇-D group wherein R₁₇ is an alkylene and D is a quaternary ammonium group;

A₁ and B₁ are polymethylene groups comprising 2 to 20 carbon atoms, which may be linear or branched, saturated or unsaturated, and which may comprise, linked to or intercalated in the main chain, at least one aromatic ring or at least one oxygen or sulfur atom or sulfoxide, sulfone, disulfide, amino, alkylamino, hydroxyl, quaternary ammonium, ureido, amide, or ester group; and

X⁻ is an anion derived from a mineral or organic acid;

A₁, R₁₃, and R₁₅ can form, with the two nitrogen atoms to which they are attached, a piperazine ring; in addition, if A₁ is a linear or branched, saturated or unsaturated alkylene or hydroxyalkylene radical, B₁ can also be (CH₂)ₙ—CO-D-OC—(CH₂)ₚ

wherein:

n and p, which may be identical or different, are integers ranging from 2 to 20,

D is chosen from:

a) a glycol residue of formula: —O-Z-O—, wherein Z is chosen from a linear and branched hydrocarbon-based radical and a group of one of the following formulae:

(CH₂—CH₂—O)ₓ—CH₂—CH₂—; and

[CH₂—CH(CH₃)—O]_{y}—CH₂—CH(CH₃)—,

wherein x and y are integers ranging from 1 to 4, representing a defined and unique degree of polymerization or any integer ranging from 1 to 4 representing an average degree of polymerization;

b) a bis-secondary diamine residue such as a piperazine derivative;

c) a bis-primary diamine residue of formula: —NH—Y—NH—, wherein Y is chosen from a linear and branched hydrocarbon-based radical, and a divalent radical of formula:

—CH2-CH2-S—S—CH2-CH2-; and

d) a ureylene group of formula: —NH—CO—NH—.

According to at least one embodiment, X⁻ is an anion such as chloride or bromide.

As disclosed herein, these polymers have, for example, a number-average molecular mass of between 1 000 and 100 000.

Polymers of this type are described, for example, in French Patent Nos. 2 320 330, 2 270 846, 2 316 271, 2 336 434, and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020.

Other polymers that may be used herein comprise repeating units of formula (a):

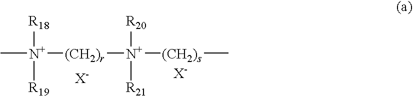

wherein R₁₈, R₁₉, R₂₀, and R₂₁, which may be identical or different, are chosen from alkyl and hydroxyalkyl radicals comprising 1 to 4 carbon atoms, n and p are integers ranging from 2 to 20, and X⁻ is an anion derived from a mineral or organic acid.

According to at least one embodiment of the present disclosure, the compound of formula (a) wherein R₁₈, R₁₉, R₂₀, and R₂₁ are methyl radicals and r is 3, is 6, and X is Cl, which is known as Hexadimethrine chloride according to the INCI (CTFA) nomenclature may be used
(5) polyquaternary ammonium polymers comprising units of formula (XV):

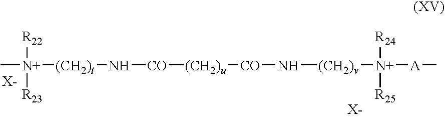

wherein:

R₂₂, R₂₃, R₂₄, and R₂₅, which may be identical or different, are chosen from hydrogen atoms and methyl, ethyl, propyl, β-hydroxyethyl, β-hydroxypropyl, and —CH₂CH₂(OCH₂CH₂)ₚOH radicals,

wherein p is an integer ranging from 0 to 6, with the proviso that R₂₂, R₂₃, R₂₄, and R₂₅ are not simultaneously hydrogen atoms,

t and u, which may be identical or different, are integers ranging from 1 to 6,

v is an integer ranging from 0 to 34,

X⁻ is an anion such as a halide,

A is chosen from a dihalide radical and —CH₂—CH₂—O—CH₂—CH₂—.

Such compounds are described, for instance, in European Patent Application No. 0 122 324.

Non-limiting examples that may be mentioned include the products MIRAPOL® A15, MIRAPOL® AD1, MIRAPOL® AZ1 and MIRAPOL® 175 sold by the company Miranol.

Other cationic polymers that may be used according to the present disclosure include, but are not limited to, polyalkyleneimines, such as polyethyleneimines, polymers comprising vinylpyridine or vinylpyridinium units, condensates of polyamines and of epichlorohydrin, quaternary polyureylenes, and chitin derivatives.

According to at least one embodiment, cationic cyclopolymers are used, for example, the dimethyldiallylammonium chloride homopolymers sold under the name MERQUAT 100 by the company Nalco (and its homologues of low weight-average molar mass), and polyethyleneimines, and mixtures thereof.

According to the present disclosure, the at least one cationic polymer (iii) with a cationic charge density of greater than 4 meq./g may be present in an amount ranging from 0.001% to 10% by weight, for example from 0.005% to 5% by weight, and further for example from 0.01% to 3% by weight, relative to the total weight of the final composition.

In at least one embodiment, the compositions also comprise at least one non-amino silicone and/or at least one other agent that is beneficial to keratin material, such as the hair, for example, esters of C₁-C₃₀ carboxylic acids and C₁-C₃₀ mono- or polyhydroxylated alcohols, plant, animal, mineral, or synthetic oils, waxes, ceramides, and pseudoceramides.

The additional non-amino silicones that may be used in accordance with the present disclosure are, for example, polyorganosiloxanes that are insoluble in the composition and may be in the form of oils, waxes, resins, or gums.

These additional organopolysiloxanes are defined, for instance, in Walter Noll's “Chemistry and Technology of Silicones” (1968) Academic Press. They may be volatile or non-volatile.

When they are volatile, the additional silicones may be chosen, for instance, from those having a boiling point of between 60° C. and 260° C., such as:

(i) cyclic silicones comprising from 3 to 7 silicon atoms, such as from 4 to 5 silicon atoms. These are, for example, octamethylcyclotetrasiloxane sold under the name VOLATILE SILICONE 7207 by Union Carbide or SILBIONE 70045 V 2 by Rhodia Chimie, decamethylcyclopentasiloxane sold under the name VOLATILE SILICONE 7158 by Union Carbide, and SILBIONE 70045 V 5 by Rhodia Chimie, and mixtures thereof.

Non-limiting mention may also be made of cyclocopolymers of the dimethylsiloxanes/methylalkylsiloxane type, such as VOLATILE SILICONE FZ 3109 sold by the company Union Carbide, with the chemical structure:

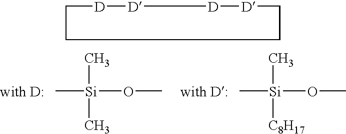

Mention may also be made, by way of non-limiting example, of mixtures of cyclic silicones with organosilicon compounds, such as the mixture of octamethylcyclotetrasiloxane and tetratrimethylsilyipentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones comprising 2 to 9 silicon atoms and having a viscosity of less than or equal to 5×10⁻⁶ m²/s at 25° C., for example, decamethyltetrasiloxane sold, for instance, under the name SH 200 by the company Toray Silicone. Silicones belonging to this category are also described, for instance, in the article published in Cosmetics and Toiletries, Vol. 91, Jan. 76, pp. 27-32, Todd & Byers “Volatile Silicone Fluids for Cosmetics.”

These additional silicones are chosen, for instance, from polyalkylsiloxanes, such as polydimethylsiloxanes comprising trimethylsilyl end groups having a viscosity of from 5×10⁻⁶ to 2.5 m²¹s at 25° C., such as 1×10⁻⁵ to 1 m²/s. The viscosity of the silicones is measured, for example, at 25° C. according to ASTM standard 445 Appendix C.

Among these polyalkylsiloxanes, mention may be made, in a non-limiting manner, of the following commercial products:

the SILBIONE oils of the 47 and 70 047 series or the MIRASIL oils sold by Rhodia Chimie, such as, the oil 70 047 V500 000;

the oils of the MIRASIL series sold by the company Rhodia Chimie;

the oils of the 200 series from the company Dow Corning, such as DC200 with a viscosity of 60 000 cSt; and

the VISCASIL oils from General Electric and certain oils of the SF series (SF 96, SF 18) from General Electric.

Non-limiting mention may also be made of polydimethylsiloxanes comprising dimethylsilanol end groups (Dimethiconol according to the CTFA name) such as the oils of the 48 series from the company Rhodia Chimie.

Non-limiting examples of polyalkylsiloxanes include poly(C₁-C₂₀)alkylsiloxanes, for example, the products sold under the names ABIL WAX 9800 and 9801 by the company Goldschmidt.

The silicone gums which can be used as additive are, for example, polydiorganosiloxanes having high number-average molecular masses of between 200 000 and 1 000 000, used alone or as a mixture in a solvent. This solvent can be chosen, for example, from volatile silicones, polydimethylsiloxane (PDMS) oils, polyphenylmethyl-siloxane (PPMS) oils, isoparaffins, polyisobutylenes, methylene chloride, pentane, dodecane, and tridecanes, and mixtures thereof.

Non-limiting mention may be made, for example, of the following products:

polydimethylsiloxane; and

polydimethylsiloxane/methylvinyisiloxane gums.

Suitable products that may be used herein, are mixtures such as:

1) mixtures formed from a polydimethylsiloxane gum hydroxylated at the end of the chain (referred to as Dimethiconol according to the CTFA nomenclature) and from a cyclic polydimethylsiloxane (referred to as Cyclomethicone according to the CTFA nomenclature), such as the product Q2 1401 sold by the company Dow Corning;

2) mixtures formed from a polydimethylsiloxane gum with a cyclic silicone, such as the product SF 1214 SILICONE FLUID from the company General Electric; this product is an SE 30 gum of molecular weight 500 000, dissolved in SF 1202 SILICONE FLUID (decamethylcyclopentasiloxane); and

3) mixtures of two PDMSs of different viscosities, such as a PDMS gum and a PDMS oil, for example, the products SF 1236 and CF1241 from the company General Electric.

The organopolysiloxane resins that may be used as additive are crosslinked siloxane systems comprising the following units:

R₂SiO_{2/2}, R₃SiO_{1/2}, RSiO_{3/2}, and SiO_{4/2} wherein R is chosen from C₁-C₁₆ hydrocarbon groups and a phenyl group. In at least one embodiment, R is chosen from C₁-C₄ lower alkyl radicals, for instance, methyl or a phenyl radical.

Among these resins, non-limiting n mention may be made of the product sold, for example, under the name DOW CORNING 593 or silicones of dimethyl/trimethyl siloxane structure sold, for instance, under the names SILICONE FLUID SS 4230 and SS 4267 by the company General Electric.

Mention may also be made in a non-limiting manner of the trimethyl siloxysilicate type resins sold, for example, under the names X22-4914, X21-5034, and X21-5037 by the company Shin-Etsu.

According to the present disclosure, all the silicones may be used in unmodified form or in the form of solutions, emulsions, nanoemulsions, or microemulsions.

In at least one embodiment, the silicones are chosen from:
non-volatile silicones from the family of polyalkylsiloxanes comprising trimethylsilyl end groups, such as oils with a viscosity between 0.2 and 2.5 m²/s at 25° C., such as the oils of the DC200 series from Dow Corning, for example, the product of viscosity 60 000 cSt, of the SILBIONE 70047 and 47 series, such as the oil 70 047 V 500 000, sold by the company Rhodia Chimie, and polyalkylsiloxanes comprising dimethylsilanol end groups, such as dimethiconols or polyalkylarylsiloxanes, such as the oil SLBIONE 70641 V 200 sold by the company Rhodia Chimie.

According to the present disclosure, the additional silicones or the other additional beneficial agents can be present in an amount ranging from 0.001% to 20% by weight, such as from 0.01% to 10% by weight, and further for example from 0.1% to 5% by weight, relative to the total weight of the final composition.

In at least one embodiment, the compositions of the present disclosure also comprise at least one surfactant, which is present in an amount ranging from approximately 0.01% to 50% by weight, for example, from 0.1% to 40% by weight, and further for example, from 0.5% to 30% by weight, relative to the total weight of the composition.

The at least one surfactant may be chosen, for example, from anionic, amphoteric, nonionic, cationic surfactants, and mixtures thereof.

Suitable surfactants include, but are not limited to:

(i) Anionic Surfactants:

Non-limiting examples of anionic surfactants include salts (such as alkaline salts, for example, sodium salts, ammonium salts, amine salts, amino alcohol salts, and magnesium salts of the following compounds: alkyl sulfates, alkyl ether sulfates, alkylamidoether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates, wherein the alkyl or acyl radical of all of these various compounds comprise, in at least one embodiment, from 8 to 24 carbon atoms, and the aryl radical is a phenyl or benzyl group. Among the anionic surfactants, non-limiting mention may also be made of fatty acid salts such as the salts of oleic, ricinoleic, palmitic and stearic acids, coconut oil acid, or hydrogenated coconut oil acid; acyl lactylates wherein the acyl radical comprises 8 to 20 carbon atoms. Mention may also be made of weakly anionic surfactants such as alkyl-D-galactosiduronic acids and their salts, as well as polyoxyalkylenated (C₆-C₂₄) alkyl ether carboxylic acids, polyoxyalkylenated (C₅-C₂₄) alkylaryl ether carboxylic acids, polyoxyalkylenated (C₆-C₂₄) alkylamido ether carboxylic acids, and their salts, for example, those comprising from 2 to 50 ethylene oxide groups, and mixtures thereof.

According to at least one embodiment, alkyl sulfate salts, alkyl ether sulfate salts, and mixtures thereof are used as anionic surfactants.

(ii) Nonionic Surfactants:

The nonionic surfactants are compounds that are well known (see, for example, “Handbook of Surfactants” by M. R. Porter, published by Blackie & Son (Glasgow and London), 1991, pp. 116-178) and, in the context of the present disclosure, their nature is not a critical feature. Thus, they can be chosen, for example, from polyethoxylated, polypropoxylated, and polyglycerolated fatty acids, alkylphenols, α-diols or alcohols having a fatty chain comprising, for example, 8 to 18 carbon atoms, it being possible for the number of ethylene oxide or propylene oxide groups to range, for instance, from 2 to 50 and for the number of glycerol groups to range, for example, from 2 to 30. Non-limiting mention may also be made of copolymers of ethylene oxide and of propylene oxide, condensates of ethylene oxide, and of propylene oxide with fatty alcohols; polyethoxylated fatty amides having, in at least one embodiment, from 2 to 30 mol of ethylene oxide, polyglycerolated fatty amides comprising on average 1 to 5, such as 1.5 to 4, glycerol groups; polyethoxylated fatty amines having, for instance, 2 to 30 mol of ethylene oxide; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides, and N-acylaminopropylmorpholine oxides. In at least one embodiment, the nonionic surfactants alkylpolyglycosides and the oxyethylenated fatty acid esters of sorbitan having from 2 to 30 mol of ethylene oxide are used.

In another embodiment of the present disclosure, the compositions comprise sorbitan laurate oxyethylenated with 4 mol of ethylene oxide (TWEEN 21 from Uniqema).

(iii) Amphoteric Surfactants:

The amphoteric surfactants that may be used herein include, but are not limited to, aliphatic secondary and tertiary amine derivatives wherein the aliphatic radical is a linear or branched chain comprising 8 to 22 carbon atoms and comprising at least one water-soluble anionic group (for example, carboxylate, sulfonate, sulfate, phosphate, and phosphonate); non-limiting mention may also be made of (C₈-C₂₀)alkylbetaines, sulfobetaines, (C₈-C₂₀)alkylamido(C₁-C₆)alkylbetaines, and (C₈-C₂₀)alkylamido(C₁-C₆)alkyl-sulfobetaines.

Among the (C₈-C₂₀)alkylamido(C₁-C₆)alkylbetaines that may be mentioned include, but are not limited to the cocoamidopropylbetaine sold, for instance by Goldschmidt under the name TEGOBETAINE F50.

Among the amine derivatives, non-limiting mention may be made, for example, of the products sold under the name MIRANOL, as described in U.S. Pat. Nos. 2,528,378 and 2,781,354 and having the structures:

R₂—CONHCH₂CH₂—N(R₃)(R₄)(CH₂COO—) (2)

wherein: R₂ is chosen from an alkyl radical derived from an acid R₂—COOH present in hydrolysed coconut oil, heptyl, nonyl, and undecyl radicals, R₃ is a β-hydroxyethyl group and R₄ is a carboxymethyl group; and

R₅—CONHCH₂CH₂—N(B)(C) (3)

wherein:

B is —CH₂CH₂OX′, C is —(CH₂)ₙ—Y′, with z=1 or 2,

X′ is chosen from —CH₂CH₂—COOH and a hydrogen atom,

Y′ is chosen from —COOH and a —CH₂—CHOH—SO₃H radical, and

R₅ is chosen from an alkyl radical of an acid R₉—COOH present in coconut oil or in hydrolysed linseed oil, and alkyl radicals, for example, a C₇, C₉, C₁₁, and C₁₃ alkyl radical, a C₁₇ alkyl radical and its iso form, and an unsaturated C₁₇ radicals; and R₉ is chosen from alkyl radicals derived from flax and coco.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

By way of non-limiting example, mention may be made of the cocoamphodiacetate sold under the trade name MIRANOL C2M concentrated by the company Rhodia Chimie.

(iv) The cationic surfactants may be chosen from:

A) the quaternary ammonium salts of formula (XVI):

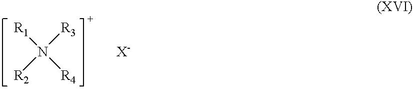

wherein X is an anion chosen from halides (chloride, bromide, or iodide), (C₂-C₆)alkyl sulfates, such as methyl sulfate, phosphates, alkyl, and alkylaryl sulfonates, and anions derived from organic acid, such as acetate or lactate, and

a) the radicals R₁ to R₃, which may be identical or different, are chosen from linear and branched C₁-C₄ aliphatic radicals, and aromatic radicals, such as aryl or alkylaryl. The aliphatic radicals can comprise heteroatoms such as oxygen, nitrogen, sulfur, and halogens. The aliphatic radicals are chosen, for example, from alkyl, alkoxy, and alkylamide radicals,

R₄ is chosen from a linear and branched C₁₆-C₃₀ alkyl radical. The cationic surfactant is, for instance, a behenyltrimethylammonium salt (for example, chloride).

b) the radicals R₁ and R₂, which may be identical or different, are chosen from linear and branched C₁-C₄ aliphatic radicals, and aromatic radicals, such as aryl or alkylaryl. The aliphatic radicals can comprise heteroatoms such as oxygen, nitrogen, sulfur, and halogens. The aliphatic radicals are chosen, for example, from alkyl, alkoxy, alkylamide, and hydroxyalkyl radicals comprising from about 1 to 4 carbon atoms;

R₃ and R₄, which may be identical or different, are chosen from linear and branched C₁₂-C₃₀ alkyl radicals, wherein the radicals comprise at least one ester or amide function.

R₃ and R₄ are chosen, for example, from (C₁₂-C₂₂)alkylamido(C₂-C₆)alkyl and (C₁₂-C₂₂)allylacetate radicals.

The cationic surfactant is, for instance, a stearamidopropyldimethyl(myristyl acetate)ammonium salt (for example, chloride).

B)—the quaternary ammonium salts of imidazolinium, such as that of formula (XVII):

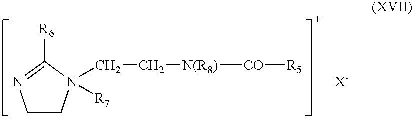

wherein R₅ is chosen from a C₈-C₃₀ alkenyl and alkyl radical, for example fatty acid derivatives of tallow, R₆ is chosen from a hydrogen atom, a C₁-C₄ alkyl radical, and a C₈-C₃₀ alkenyl and alkyl radical, R₇ is chosen from a C₁-C₄ alkyl radical, R₈ is chosen from a hydrogen atom and a C₁-C₄ alkyl radical, and X is an anion chosen from halides, phosphates, acetates, lactates, alkyl sulfates, alkyl sulfonates, and alkylaryl sulfonates. In at least one embodiment, R₅ and R₆ are a mixture of C₁₂-C₂₁ alkenyl or alkyl radicals, such as fatty acid derivatives of tallow, R₇ is methyl and R₈ is a hydrogen atom. Such a product is, for example, QUATERNIUM-27 (CTFA 1997) or QUATERNIUM-83 (CTFA 1997), which are sold under the names REWOQUAT W75, W90, W75PG and W75HPG by the company Witco,

C)—the diquaternary ammonium salts of formula (XVIII):

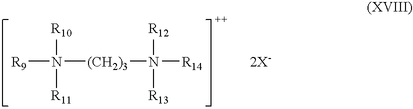

wherein R₉ is chosen from a C₁₆-C₃₀ aliphatic radical, R₁₀, R₁₁, R₁₂, R₁₃, and R₁₄, which may be identical or different, are chosen from hydrogen atoms and C₁-C₄ alkyl radicals, and X is an anion chosen from a halides, acetates, phosphates, nitrates, and methyl sulfates. Such diquaternary ammonium salts, for instance, comprise propanetallowediammonium dichloride;

D)—the quaternary ammonium salts comprising at least one ester function of formula (XIX):

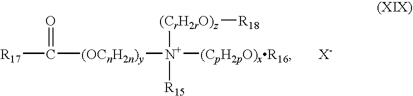

wherein
R₁₅ is chosen from a C₁-C₆ alkyl radical and a C₁-C₆ hydroxyalkyl, and a dihydroxyalkyl radical;
R₁₆ is chosen from:

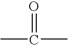

a radical R₁₉
R₂₀, wherein R₂₀ is chosen from linear and branched, saturated and unsaturated C₁-C₂₂ hydrocarbon radicals, and
a hydrogen atom,
R₁₈ is chosen from:
a radical R₂₁

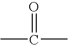

R₂₂, wherein R₂₂ is chosen from linear and branched, saturated and unsaturated C₁-C₆ hydrocarbon radicals, and
a hydrogen atom,
R₁₇, R₁₉, and R₂₁, which may be identical or different, are chosen from linear and branched, saturated and unsaturated C₇-C₂₁ hydrocarbon radicals;
n, p, and r, which may be identical or different, are integers ranging from 2 to 6;
y is an integer ranging from 1 to 10;
x and z, which may be identical or different, are integers ranging from 0 to 10; and
X⁻ is a simple or complex, organic or inorganic anion; with the proviso that the sum x+y+z is from 1 to 15, that when x is 0, then R₁₆ is R₂₀, and that when z is 0, then R₁₈ is R₂₂.

Useful ammonium salts according to the present disclosure include, but are not limited to, those of formula (XIX) wherein:

R₁₅ is a methyl or ethyl radical;

x and y are 1;

z is 0 or 1;

n, p, and r are 2;

R₁₆ is chosen from:
a radical R₁₉

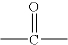

methyl, ethyl, and C₁₄-C₂₂ hydrocarbon radicals, and
a hydrogen atom;

R₁₇, R₁₉, and R₂₁, which may be identical or different, are chosen from linear and branched, saturated and unsaturated C₇-C₂₁ hydrocarbon radicals; and

R₁₈ is chosen from:
a radical R₂₁

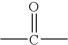

and a hydrogen atom.

Such compounds are sold, for example, under the names DEHQUART by the company Cognis, STEPANQUAT by the company Stepan, NOXAMIUM by the company Ceca, and REWOQUAT WE 18 by the company Rewo-Witco, Suitable quaternary ammonium salts include, but are not limited to, behenyltrimethylammonium chloride and stearamidopropylmethyl(myristyl acetate)ammonium chloride, sold, for instance, under the name CERAPHYL 70 by the company Van Dyk, and QUATERNIUM-27 or QUATERNIUM-83 sold by the company Witco.

In at least one embodiment of the present disclosure, the compositions may comprise mixtures of surfactants, for example, mixtures of anionic surfactants, mixtures of anionic surfactants and of amphoteric, cationic and nonionic surfactants, or mixtures of cationic surfactants with nonionic or amphoteric surfactants. In another embodiment, a mixture comprising at least one anionic surfactant and at least one amphoteric surfactant may be used.

The composition of the present disclosure may also comprise at least one additive chosen from thickeners, fragrances, nacreous agents, preserving agents, silicone and non-silicone sunscreens, anionic and nonionic non-silicone polymers, non-cationic proteins, non-cationic protein hydrolysates, 18-methyl eicosanoic acid, hydroxy acids, vitamins, provitamins such as panthenol, and any other additive conventionally used in cosmetics that does not affect the properties of the compositions as disclosed herein. The compositions, in accordance with the present disclosure may also comprise from 0% to 5% of nacreous or opacifying agents that are well known in the prior art, such as sodium or magnesium palmitate, sodium or magnesium stearate or hydroxystearate, fatty-chain acyl derivatives such as ethylene glycol or polyethylene glycol monostearates or distearates, fatty-chain ethers, for example, distearyl ether or 1-(hexadecyloxy)-2-octadecanol, and fatty alcohols, such as stearyl alcohol, cetyl alcohol, or behenyl alcohol, and mixtures thereof.

The at least one additive optionally present in the compositions disclosed herein may be present in an amount ranging from 0.001% to 20% by weight, relative to the total weight of the composition. The precise amount of each additive is readily determined on the basis of its nature and its function by a person skilled in the art. According to the present disclosure, the cosmetically acceptable medium may consist solely of water or of a mixture of water and a cosmetically acceptable solvent such as a C₁-C₄ lower alcohol, for instance, ethanol, isopropanol, tert-butanol, and n-butanol; alkylene glycols, for instance propylene glycol, and glycol ethers. In at least one embodiment, the composition comprises water in an amount ranging from 50% to 95% by weight, relative to the total weight of the composition, such as from 60 to 90% by weight.

The compositions according to the present disclosure have a final pH generally of between 3 and 10. In one embodiment, this pH is between 4 and 8. Adjusting the pH to the desired value may be performed conventionally by adding a base (organic or mineral base) to the composition, for example aqueous ammonia or a primary, secondary or tertiary (poly)amine, for instance monoethanolamine, diethanolamine, triethanolamine, isopropanolamine or 1,3-propanediamine, or by adding a mineral or organic acid, such as a carboxylic acid, for example, citric acid.

The compositions, in accordance with the present disclosure, may be used, for example, for washing or treating keratin materials such as the hair, the skin, the eyelashes, the eyebrows, the nails, the lips or the scalp, and in at least one embodiment, the hair.

The compositions disclosed herein may also be detergent compositions such as shampoos, shower gels, and bubble baths and comprise at least one washing base, which is generally aqueous.

The at least one surfactant constituting the washing base may be chosen alone or as mixtures of anionic, amphoteric, nonionic, and cationic surfactants as defined above. The washing base comprises at least one detergent surfactant.

In at least one embodiment of the present disclosure, the compositions comprise at least one anionic surfactant or mixtures of at least one anionic surfactant and of at least one amphoteric surfactant or of at least one nonionic surfactant.

In another embodiment, a mixture comprising at least one anionic surfactant and at least one amphoteric surfactant may be used.

Use may be made, for example, of an anionic surfactant chosen from sodium, triethanolamine or ammonium (C₁₂-C₁₄)alkyl sulfates, sodium, triethanolamine or ammonium (C₁₂-C₁₄)alkyl ether sulfates oxyethylenated comprising 2.2 mol of ethylene oxide, sodium cocoyl isethionate and sodium α-(C₁₄-C₁₆)olefin sulfonate, and mixtures thereof with:

either an amphoteric surfactant such as the amine derivatives known as disodium cocoamphodipropionate or sodium cocoamphopropionate sold, for example, by the company Rhodia Chimie under the trade name MIRANOL C2M Conc. as an aqueous solution comprising 38% active material, or under the name MIRANOL C32;

or an amphoteric surfactant of zwitterionic type, such as alkylbetaines or alkylamidobetaines such as the cocobetaine sold, for instance, under the name DEHYTON AB 30 as an aqueous solution comprising 32% AM by the company Cognis, or the cocoamidopropylbetaine sold, for example, by Goldschmidt under the name TEGOBETAINE F50.

The quantity and quality of the washing base is chosen in order to give the final composition satisfactory foaming power and/or detergent power.

These detergent compositions are, for example, foaming compositions and the foaming power of the compositions disclosed herein, characterized by a foam height, may be greater than 75 mm, for example, greater than 100 mm, measured according to the modified Ross-Miles method (NF T 73-404/IS696). The modifications to the method are the following:

The measurement is performed at a temperature of 22° C. with osmosed water. The concentration of the solution is 2 g/l. The height of the drop is 1 m. The amount of composition that is dropped is 200 ml. These 200 ml of composition fall into a measuring cylinder 50 mm in diameter and containing 50 ml of the test composition. The measurement is taken 5 minutes after stopping the flow of the composition.

Thus, according to the present disclosure, the detergent surfactants may be present in an amount ranging from 3% to 50% by weight, such as from 6% to 35% by weight, and even further for example from 8% to 25% by weight, relative to the total weight of the final composition.

Another aspect of the present disclosure is a cosmetic process for treating keratin materials such as the skin or the hair, comprising applying to the keratin materials a cosmetic composition as defined above, and then optionally rinsing it out with water after an optional leave-in time.

Thus, the process as disclosed herein allows for the maintenance of the hairstyle and the treatment, care, washing, or removal of makeup of the skin, the hair, or any other keratin material.

The compositions of the present disclosure may also be in the form of rinse-out or leave-in conditioners, permanent-waving, hair-straightening, dyeing, or bleaching compositions, or in the form of rinse-out compositions to be applied before or after dyeing, bleaching, permanent-waving, or straightening the hair or between the two steps of a permanent-waving or hair-straightening operation.

When the composition is in the form of a conditioner, which may be a rinse-out conditioner, it comprises, in at least one embodiment, at least one cationic surfactant, wherein the concentration of said at least one cationic surfactant may range from 0.1% to 10% by weight, such as from 0.5% to 5% by weight, relative to the total weight of the composition.

The compositions of the present disclosure may also be in the form of washing compositions for the skin, such as bath or shower solutions or gels or makeup-removing products.

The compositions disclosed herein may also be in the form of aqueous or aqueous-alcoholic lotions for skincare and/or haircare.

The cosmetic compositions according to the present disclosure may be in the form of a gel, a milk, a cream, an emulsion, a thickened lotion or a mousse and may be used for the skin, the nails, the eyelashes, the lips and in at least one embodiment, the hair.

The compositions may be conditioned in various forms, such as in vaporizers, pump-dispenser bottles or in aerosol containers to allow an application of the composition in vaporized form or in the form of a mousse. Such conditioning forms are indicated, for example, when it is desired to obtain a spray, a lacquer, or a mousse for treating keratin material, such as the hair.

As disclosed herein, the percentages stated are by weight.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The examples that follow are intended to illustrate the present disclosure without, however, being limiting in nature.

In the examples, AM means active material.

### EXAMPLE 1

A hair conditioner composition, in accordance with the present disclosure, was prepared:

| Copolymer of acrylic or methacrylic acid esters, | 0.2 | g AM |
|---|---|---|
| of di(C1-4)alkylamino(C1-6)alkyl methacrylate, of | | |
| PEG/PPG-30/5 allyl ether, of C10-30 PEG 20-25 alkyl | | |
| ether methacrylate, and of C2-6 hydroxyalkyl | | |
| methacrylate crosslinked with ethylene glycol | | |
| dimethacrylate as a 20% emulsion in water | | |
| (CARBOPOL Aqua CC from Noveon) | | |
| Dimethyldiallylammonium chloride homopolymer | 0.96 | g AM |
| as a 40% solution in water (MERQUAT 100 from | | |
| Nalco) | | |
| Quaternium-80 as a solution comprising 50% | 0.375 | g AM |
| AM in propylene glycol (ABIL QUAT 3272 from | | |
| Goldschmidt) | | |
| Oxyethylenated sorbitan monolaurate (20 EO) | 1.6 | g |
| (TWEEN 20 from UNIQEMA) | | |
| Cetyltrimethylammonium chloride as an | 0.5 | g AM |
| aqueous solution comprising 25% active material | | |
| (ARQUAD 16-25 LO from AKZO NOBEL) | | |
| Preserving agents | qs | |
| Fragrance | 0.2 | g |
| Lactic acid qs | pH | 4 |
| Deionized water qs | 100 | g |

This stable composition of pleasant texture was applied to highly sensitized hair. It was easily removed during rinsing. The cosmetic properties of the treated hair (disentangling, smoothness and suppleness) were excellent and homogeneous from the roots to the ends of the hair.

### EXAMPLE 2

A hair conditioner composition in accordance with the present disclosure, was prepared:

| Copolymer of acrylic or methacrylic acid esters, | 0.2 | g AM |
|---|---|---|
| of di(C1-4)alkylamino(C1-6)alkyl methacrylate, of | | |
| PEG/PPG-30/5 allyl ether, of C10-30 PEG 20-25 alkyl | | |
| ether methacrylate, and of C2-6 hydroxyalkyl | | |
| methacrylate crosslinked with ethylene glycol | | |
| dimethacrylate as a 20% emulsion in water | | |
| (CARBOPOL AQUA CC from Noveon) | | |
| Dimethyldiallylammonium chloride homopolymer | 0.96 | g AM |
| as a 40% solution in water (MERQUAT 100 from | | |
| Nalco) | | |
| Polydimethylsiloxane containing aminoethyl | 0.5 | g AM |
| iminopropyl groups, as a microemulsion at 17% in | | |
| (WACKER-BELSIL AMD LOG 1 from Wacker) | | |
| Oxyethylenated sorbitan monolaurate (20 EO) | 1.6 | g |
| (TWEEN 20 from Uniqema) | | |
| Behenyltrimethylammonium chloride as a 72.9% | 0.7 | g AM |
| solution in water/isopropanol (GENAMIN KDMP from | | |
| Clariant) | | |
| Preserving agents | qs | |
| Fragrance | 0.2 | g |
| Lactic acid qs | pH | 4 |
| Deionized water qs | 100 | g |

This stable composition of pleasant texture was applied to highly sensitized hair. It was easily removed during rinsing. The cosmetic properties of the treated hair (disentangling, smoothness and suppleness) were excellent and homogeneous from the roots to the ends of the hair.

What is claimed is:

1. A cosmetic composition, comprising, in a cosmetically acceptable medium:
(i)—at least one cationic polymer, which is a product of polymerization of a monomer mixture comprising:
a) at least one vinyl monomer substituted with at least one amino group, and
b) at least one hydrophobic nonionic vinyl monomer chosen from formula (I) and (II):
CH₂═C(X)Z, and (I)
CH₂═CH—OC(O)R; (II)
wherein:
X is chosen from a hydrogen atom and a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R independently is a C₁-C₃₀ alkyl group; and
c) at least one associative vinyl monomer,
(ii)—at least one amino silicone, and
(iii)—at least one cationic polymer other than the at least one cationic polymer (i) and the at least one amino silicone (ii) and having a cationic charge density of greater than or equal to 4 meq./g.

2. The cosmetic composition according to claim 1, wherein the at least one vinyl monomer substituted with at least one amino group is chosen from:
mono(C₁-C₄)alkylamino(C₁-C₈)alkyl (meth)acrylates,
di(C₁-C₄)alkylamino(C₁-C₈)alkyl (meth)acrylates,
mono(C₁-C₄)alkylamino(C₁-C₈)alkyl(meth)acrylamides,
di(C₁-C₄)alkylamino(C₁-C₈)alkyl(meth)acrylamides,
heterocyclic (meth)acrylamides comprising a nitrogen atom,
heterocyclic (meth)acrylates comprising a nitrogen atom,
and mixtures thereof.

3. The cosmetic composition according to claim 2, wherein the at least one vinyl monomer substituted with at least one amino group is chosen from:
mono- and di(C₁-C₄ alkyl)amino(C₁-C₄ alkyl) (meth)acrylates;
mono- and di(C₁-C₄ alkyl)amino(C₁-C₄ alkyl)(meth)acrylamides;
(meth)acrylamides and (meth)acrylates with a heterocyclic group comprising a nitrogen atom; and
nitrogenous heterocycles comprising at least one vinyl group.

4. The cosmetic composition according to claim 1, wherein the at least one vinyl monomer substituted with least one amino group is present in amount ranging from 10% to 70% by weight, relative to the total weight of the monomer mixture.

5. The cosmetic composition according to claim 1, wherein the at least one hydrophobic nonionic vinyl monomer is chosen from C₁-C₃₀ alkyl (meth)acrylates, (C₁-C₃₀ alkyl)(meth)acrylamides, styrene, substituted styrenes, vinyl esters, unsaturated nitriles, and unsaturated silanes.

6. The cosmetic composition according to claim 1, wherein the at least one hydrophobic nonionic vinyl monomer is present in an amount ranging from 20% to 80% by weight, relative to the total weight of the monomer mixture.

7. The cosmetic composition according to claim 1, wherein the at least one associative vinyl monomer is chosen from compounds of formula (III): 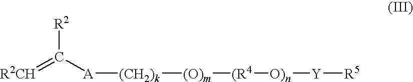
wherein:
each R² is independently chosen from a hydrogen atom, a methyl group, a —C(O)OH group, and a —C(O)OR³ group;
R³ is a C₁-C₃₀ alkyl;
A is chosen from —CH₂C(O)O—, —C(O)O—, —O—, CH₂O, —NHC(O)NH—, —C(O)NH—, —Ar—(CE₂)_{z}—NHC(O)O—, —Ar—(CE₂)_{z}-NHC(O)NH—, and —CH₂CH₂—NHC(O)— groups;
Ar is a divalent aryl group;
E is chosen from a hydrogen atom and a methyl group;
z is an integer ranging from 0 to 1;
k is an integer ranging from 0 to 30;
m is an integer ranging from 0 to 1, with the provisos that when k is 0, then m is 0, and when k is an integer ranging from 1 to 30, then m is 1;
(R⁴—O)ₙ is a polyoxyalkylene, which is a homopolymer, a random copolymer or a block copolymer, comprising C₂-C₄ oxyalkylene units;
R⁴ is chosen from C₂H₄, C₃H₆, C₄H₈, and mixtures thereof;
n is an integer ranging from 5 to 250;
Y is chosen from —R⁴O—, —R⁴NH—, —C(O)—, —C(O)NH—, R⁴NHC(O)NH—, and —C(O)NHC(O)—; and
R⁵ is chosen from substituted and unsubstituted linear C₈-C₄₀ alkyl groups, branched C₈-C₄₀ alkyl groups, C₈-C₄₀ alicyclic groups, phenyls substituted with a C₂-C₄₀ alkyl group, C₂-C₄₀ alkyl groups substituted with an aryl group, and C₈-C₈₀ complex esters, wherein the alkyl group R⁵ optionally comprises at least one substituent chosen from hydroxyl, alkoxy, and halo groups.

8. The cosmetic composition according to claim 7, wherein the at least one associative vinyl monomer is chosen from polyethoxylated cetyl (meth)acrylates, polyethoxylated cetearyl (meth)acrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-tris(1′-phenylethyl)phenyl (meth)acrylates, polyethoxylated hydrogenated castor oil (meth)acrylates, polyethoxylated canola (meth)acrylates, polyethoxylated cholesteryl (meth)acrylates, and mixtures thereof, wherein the polyethoxylated portion of the monomer comprises from 5 to 100 ethylene oxide units.

9. The cosmetic composition according to claim 1, wherein the at least one associative vinyl monomer is present in an amount ranging from 0.001% to 25% by weight, relative to the total weight of the monomer mixture.

10. The cosmetic composition according to claim 1, wherein the monomer mixture further comprises at least one semi-hydrophobic vinyl surfactant monomer chosen from compounds of formula (IV) and (V): 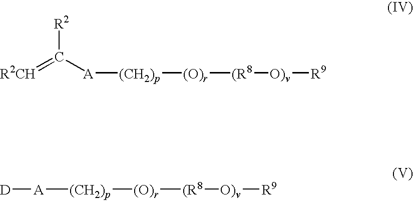
wherein:
each R⁶ independently is chosen from a hydrogen atom, a C₁-C₃₀ alkyl, —C(O)OH, and C(O)OR⁷;
R⁷ is a C₁-C₃₀ alkyl;
A is chosen from —CH₂C(O)O—, —C(O)O—, —O—, —CH₂O, —NHC(O)NH—, —C(O)NH—, —Ar—(CE₂)_{z}—NHC(O)O—, —Ar—(CE₂)_{z}-NHC(O)NH—, and —CH₂CH₂NHC(O)—;
Ar is a divalent aryl group;
E is chosen from a hydrogen atom and a methyl group;
z is an integer ranging from 0 to 1;
p is an integer ranging from 0 to 30;
r is an integer ranging from 0 to 1, with the provisos that when p is 0, then r is 0, and when p is an integer ranging from 1 to 30, then r is 1;
(R₈—O)ᵥ is a polyoxyalkylene which is a homopolymer, a random copolymer, or a block copolymer with C₂-C₄ oxyalkylene units, wherein R⁸ is chosen from C₂H₄, C₃H₆, C₄H₈, and mixtures thereof, and v is an integer ranging from 5 to 250;
R⁹ is chosen from a hydrogen atom and a C₁-C₄ alkyl;
D is chosen from C₈-C₃₀ alkenyl groups optionally substituted with a carboxyl group.

11. The cosmetic composition according to claim 10, wherein the at least one semi-hydrophobic vinyl surfactant monomer is chosen from one of the following formulae:
CH₂═CH—O(CH₂)ₐO(C₃H₆O)_{b}(C₂H₄O)_{c}H, and
CH₂═CHCH₂O(C₃H₆O)_{d}(C₂H₄O)ₑH;
wherein:
a is an integer ranging from 2 to 4;
b is an integer ranging from 1 to 10;
c is an integer ranging from 5 to 50;
d is an integer ranging from 1 to 10; and
e is an integer ranging from 5 to 50.

12. The cosmetic composition according to claim 10, wherein the at least one semi-hydrophobic vinyl surfactant monomer is present in an amount ranging from 0% to 25% by weight, relative to the total weight of the monomer mixture.

13. The cosmetic composition according to claim 1, wherein the monomer mixture also comprises at least one hydroxylated nonionic vinyl monomer.

14. The cosmetic composition according to claim 13, wherein the at least one hydroxylated nonionic vinyl monomer is chosen from C₁-C₆ hydroxyalkyl (meth)acrylates and (C₁-C₄ hydroxyalkyl)(meth)acrylamides, and mixtures thereof.

15. The cosmetic composition according to claim 14, wherein the at least one hydroxylated nonionic vinyl monomer is 2-hydroxyethyl methacrylate.

16. The cosmetic composition according to claim 13, wherein the at least one hydroxylated nonionic vinyl monomer is present in an amount ranging from 0% to 10% by weight, relative to the total weight of the monomer mixture.

17. The cosmetic composition according to claim 1, wherein the monomer mixture further comprises at least one crosslinking monomer present in an amount ranging from 0.001% to 5% by weight, relative to the total weight of the monomer mixture.

18. The cosmetic composition according to claim 17, wherein the at least one crosslinking monomer is chosen from a polyol acrylate ester comprising at least two acrylate ester groups, a polyol methacrylate ester comprising at least two methacrylate ester groups, and a combination thereof.

19. The cosmetic composition according to claim 1, wherein the monomer mixture further comprises at least one chain-transfer agent present in an amount ranging from 0% to 10% by weight, relative to the total weight of the monomer mixture.

20. The cosmetic composition according to claim 1, wherein the monomer mixture comprises, relative to the total weight of the monomer mixture:
a) from 10% to 70% by weight of at least one vinyl monomer substituted with at least one amino group,
b) from 20% to 80% by weight of at least one hydrophobic nonionic vinyl monomer,
c) from 0.001% to 25% by weight of at least one associative vinyl monomer,
d) from 0.01% to 25% by weight of at least one semi-hydrophobic vinyl surfactant monomer,
e) from 0% to 10% by weight of at least one hydroxylated nonionic vinyl monomer,
f) from 0% to 5% by weight of at least one crosslinking monomer,
g) from 0% to 10% by weight of at least one chain-transfer agent, and
h) from 0% to 2% by weight of at least one polymeric stabilizer.

21. The cosmetic composition according to claim 20, wherein the monomer mixture comprises, relative to the total weight of the monomer mixture:
a) from 20% to 60% by weight of at least one vinyl monomer substituted with at least one amino group,
b) from 20% to 70% by weight of at least one hydrophobic nonionic vinyl monomer,
c) from 0.01% to 15% by weight of at least one associative vinyl monomer,
d) from 0.1% to 10% by weight of at least one semi-hydrophobic vinyl surfactant monomer,
e) from 0.01% to 10% by weight of at least one hydroxylated nonionic vinyl monomer,
f) from 0.001% to 5% by weight of at least one crosslinking monomer,
g) from 0.001% to 10% by weight of at least one chain-transfer agent, and
h) from 0% to 2% by weight of at least one polymeric stabilizer.

22. The cosmetic composition according to claim 1, wherein the monomer mixture comprises, relative to the total weight of the monomer mixture:
a) from 20% to 50% by weight of at least one vinyl monomer substituted with at least one amino group chosen from:
3-(N,N-dimethylamino)propyl (meth)acrylate,
N′-(3-N,N-dimethylamino)propyl(meth)acrylamide,
2-(N,N-dimethylamino)ethyl (meth)acrylate,
2-(N,N-diethylamino)ethyl (meth)acrylate,
2-(tert-butylamino)ethyl (meth)acrylate,
2-(N,N-dimethylamino)propyl(meth)acrylamide, and
2-(N,N-dimethylamino)neopentyl acrylate,
b) from 50% to 65% by weight of at least one hydrophobic nonionic vinyl monomer chosen from C₁-C₃₀ alkyl esters of acrylic acid and C₁-C₃₀ alkyl esters of methacrylic acid, and mixtures thereof,
c) from 0.1% to 10% by weight of at least one associative vinyl monomer chosen from polyethoxylated cetyl methacrylates, polyethoxylated cetearyl methacrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (methacrylates, polyethoxylated lacceryl (meth)acrylates, polyethoxylated 2,4,6-tris(1′-phenylethyl)phenyl (meth)acrylates, polyethoxylated hydrogenated castor oil (meth)-acrylates, polyethoxylated canola (meth)acrylates, and polyethoxylated cholesteryl (meth)acrylates, and mixtures thereof,
d) from 0.1% to 10% by weight of at least one semi-hydrophobic vinyl surfactant monomer chosen from one of the following formulae:
CH₂═CH—O(CH₂)ₐO(C₃H₆O)_{b}(C₂H₄O)_{c}H, and
CH₂═CHCH₂O(C₃H₆O)_{d}(C₂H₄O)ₑH;
wherein:
a is integer ranging from 2 to 4;
b is an integer ranging from 1 to 10;
c is an integer ranging from 5 to 50;
d is an integer ranging from 1 to 10; and
e is an integer ranging from 5 to 50;
e) from 0% to 10% by weight of at least one hydroxylated nonionic vinyl monomer,
f) from 0% to 5% by weight of at least one crosslinking monomer,
g) from 0% to 10% by weight of at least one chain-transfer agent, and
h) from 0% to 2% by weight of at least one polymeric stabilizer.

23. The cosmetic composition according to claim 1, wherein the monomer mixture comprises:
a di(C₁-C₄ alkyl)amino(C₁-C₆ alkyl)methacrylate,
at least one C₁-C₃₀ alkyl ester of (meth)acrylic acid,
a C₁₀-C₃₀ alkyl methacrylate polyethoxylated with 20 to 30 mol of ethylene oxide,
a 30/5 polyethylene glycol/polypropylene glycol allyl ether,
a hydroxy(C₂-C₆ alkyl)methacrylate, and
an ethylene glycol dimethacrylate.

24. The cosmetic composition according to claim 1, wherein the at least one cationic polymer (i) is a thickening polymer.

25. The cosmetic composition according to claim 1, wherein the at least one cationic polymer (i) is present in an amount ranging from 0.01% to 10% by weight, relative to the total weight of the composition.

26. The cosmetic composition according to claim 1, wherein the at least one amino silicone is chosen from:
a) the compounds of formula (VI):
(R¹)ₐ(T)₃₋ₐ-Si[OSi(T)₂]ₙ-[OSi(T)_{b}(R¹)_{2-b}]ₘ—OSi(T)₃₋ₐ-(R¹)ₐ
wherein:
T is chosen from a hydrogen atom, a phenyl, a hydroxyl, a C₁-C₈ alkyl radical, and a C₁-C₈ alkoxy radical;
a is an integer ranging from 0 to 3;
b is an integer ranging from 0 to 1;
m and n are integers such that the sum (n+m) ranges from 1 to 2000, wherein n is a number ranging from 0 to 1999, and m is a number ranging from 1 to 2000;
R¹ is a monovalent radical of formula C_{q}H_{2q}L wherein q is an integer ranging from 2 to 8 and L is an optionally quaternized amino group chosen from:
—N(R²)—CH₂—CH₂—N(R²)₂;
—N(R²)₂;
N⁺(R²)₃Q⁻;
—N⁺(R²)(H)₂Q⁻;
—N⁺(R²)₂HQ⁻; and
—N(R²)—CH₂—CH₂—N⁺(R²)(H)₂Q⁻,
wherein R² is chosen from a hydrogen atom, a phenyl, a benzyl, and a saturated monovalent hydrocarbon-based radical, and Q⁻ is a halide ion;
b) the silicones of formula (IX): 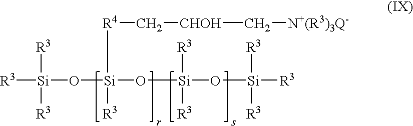
wherein:
R³ is chosen from a monovalent C₁-C₁₈ hydrocarbon-based radical;
R⁴ is chosen from a divalent hydrocarbon-based radical;
Q⁻ is a halide ion;
r is an average statistical value ranging from 2 to 20;
s is an average statistical value ranging from 20 to 200;
c) quaternary ammonium silicones; and
d) the amino silicones of formula (XI): 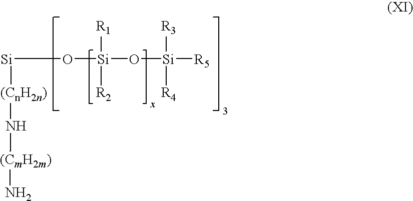
wherein:
R₁, R₂, R₃, and R₄, which may be identical or different, are chosen from C₁-C₄ alkyl radicals and phenyl groups,
R₅ is chosen from a C₁-C₄ alkyl radical and a hydroxyl group,
n is an integer ranging from 1 to 5,
m is an integer ranging from 1 to 5, and
x is an integer ranging from 2 to 500.

27. The cosmetic composition according to claim 26, wherein the at least one amino silicone corresponding to formula (VI) is chosen from compounds of formula (VII): 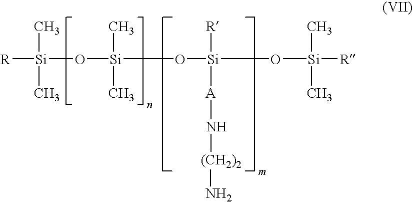
wherein R, R′, and R″, which may be identical or different, are chosen from C₁-C₄ alkyl radicals, C₁-C₄ alkoxy radicals, and OH; A is chosen from a linear and branched C₃-C₈ alkylene radical; and m and n are integers dependent on the molecular weight and whose sum is between 1 and 2000.

28. The cosmetic composition according to claim 26, wherein the at least one amino silicone of formula (VI) is the silicone corresponding to formula (VIII): 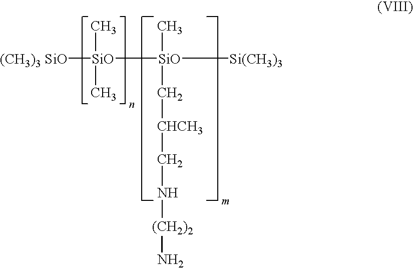
wherein n and m have the meanings given in accordance with formula (VI).

29. The cosmetic composition according to claim 1, wherein the at least one amino silicone is chosen from quaternary ammonium silicones.

30. The cosmetic composition according to claim 1, wherein the at least one amino silicone is chosen from quaternary ammonium silicones of formula (X): 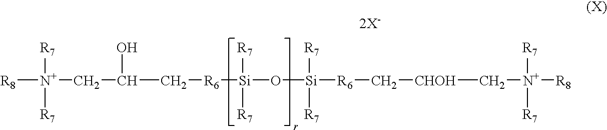
wherein:
R₇, which may be identical or different, is chosen from a C₁-C₁₈ monovalent hydrocarbon-based radical, a C₂-C₁₈ alkenyl radical, and a ring comprising 5 or 6 carbon atoms;
R₆ is chosen from a divalent hydrocarbon-based radical;
R₈, which may be identical or different, is chosen from a hydrogen atom, a C₁-C₁₈ monovalent hydrocarbon-based radical, a C₂-C₁₈ alkenyl radical, and a —R₆—NHCOR₇ radical; wherein
X⁻ is an anion; and
r is a mean statistical value ranging from 2 to 200.

31. The composition according to claim 1, wherein the at least one amino silicone (ii) is present in an amount ranging from 0.001% to 20% by weight, relative to the total weight of the composition.

32. The composition according claim 1, wherein the at least one cationic polymer (iii) with a cationic charge density of greater than 4 meq./g is chosen from:
(1) homopolymers or copolymers derived from acrylic or methacrylic esters or amides and comprising at least one of the units of the following formulae: 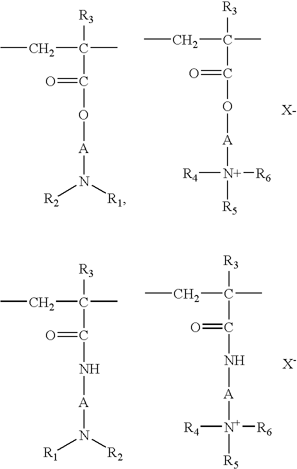
wherein:
R₃, which may be identical or different, is chosen from a hydrogen atom and a CH₃ radical;
A, which may be identical or different, is chosen from a C₁-C₆ linear and branched alkyl group and a hydroxyalkyl group comprising 1 to 4 carbon atoms;
R₄, R₅, and R₆, which may be identical or different, are chosen from C₁-C₁₈ alkyl groups and benzyl radicals;
R₁ and R₂, which may be identical or different, are chosen from hydrogen atoms and C₁-C₆ alkyl groups;
X⁻ is an anion derived from a mineral or organic acid;
(2) cyclopolymers of alkyldiallylamine or of dialkyldiallylammonium;
(3) quaternary polymers of vinylpyrrolidone and of vinylimidazole;
(4) the quaternary diammonium polymer comprising repeating units of formula (XIV): 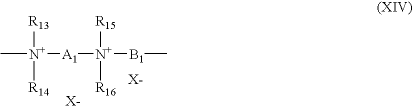
wherein:
R₁₃, R₁₄, R₁₅, and R₁₆, which may be identical or different, are chosen from aliphatic, alicyclic and arylaliphatic radicals comprising from 1 to 20 carbon atoms and lower C₁-C₆ hydroxyalkylaliphatic radicals, or R₁₃, R₁₄, R₁₅, and R₁₆, together or separately, constitute, with the nitrogen atoms to which they are attached, heterocycles optionally comprising a second heteroatom other than nitrogen, or R₁₃, R₁₄, R₁₅, and R₁₆ are chosen from linear and branched C₁-C₆ alkyl radicals substituted with a nitrile, ester, acyl, or amide group or a —CO—O—R₁₇-D or —CO—NH—R₁₇-D group wherein R₁₇ is an alkylene and D is a quaternary ammonium group;
A₁ and B₁ are chosen from polymethylene groups comprising 2 to 20 carbon atoms, which may be linear or branched, saturated or unsaturated, and which may comprise, linked to or intercalated in the main chain, at least one aromatic ring or at least one oxygen or sulfur atom or sulfoxide, sulfone, disulfide, amino, alkylamino, hydroxyl, quaternary ammonium, ureido, amide, or ester group; and
X⁻ is an anion derived from a mineral or organic acid;
A₁, R₁₃, and R₁₅ can form, with the two nitrogen atoms to which they are attached, a piperazine ring; in addition, if A₁ is chosen from a linear and branched, saturated and unsaturated alkylene radical or hydroxyalkylene radical, B₁ can also be (CH₂)ₙ—CO-D-OC—(CH₂)ₚ
wherein:
n and p, which may be identical or different, are integers ranging from 2 to 20,
D is chosen from:
a) a glycol residue of formula: —O-Z-O—, wherein Z is chosen from a linear and branched hydrocarbon-based radical and a group of one of the following formulae:
-(CH₂—CH₂—O)ₙ—CH₂—CH₂—; and
-[CH₂—CH(CH₃)—O]_{y}—CH₂—CH(CH₃)—,
wherein x and y are integers ranging from 1 to 4, representing a defined and unique degree of polymerization or any integer ranging from 1 to 4 representing an average degree of polymerization;
b) a bis-secondary diamine residue;
c) a bis-primary diamine residue of formula: —NH—Y—NH—, wherein Y is chosen from a linear and branched hydrocarbon-based radical, and a divalent radical of formula:
—CH₂—CH₂—S—S—CH₂—CH₂—; and
d) a ureylene group of formula: —NH—CO—NH—;
(5) polyquaternary ammonium polymers comprising units of formula (XV): 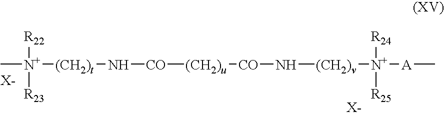
wherein:
R₂₂, R₂₃, R₂₄, and R₂₅, which may be identical or different, are chosen from hydrogen atoms, methyl, ethyl, propyl, β-hydroxyethyl, β-hydroxypropyl and —CH₂CH₂(OCH₂CH₂)ₚOH radicals,
wherein p is an integer ranging from 0 to 6, with the proviso that when R₂₂, R₂₃, R₂₄, and R₂₅ are not simultaneously hydrogen atoms,
t and u, which may be identical or different, are integers ranging from 1 to 6,
v is an integer ranging from 0 to 34,
X⁻ is an anion,
A is chosen from a dihalide radical and —CH₂—CH₂—O—CH₂—CH₂—; and
(6) polyalkyleneimines, polymers comprising vinylpyridine or vinylpyridinium units, condensates of polyamines and of epichlorohydrin, quaternary polyureylenes, and chitin derivatives.

33. The cosmetic composition according to claim 32, wherein the at least one cationic polymer (iii) with a cationic charge density of greater than 4 meq./g is chosen from cationic cyclopolymers, polyalkyleneimines, and mixtures thereof.

34. The cosmetic composition according to claim 1, wherein the at least one cationic polymer (iii) with a cationic charge density of greater than 4 meq./g is chosen from homopolymers and copolymers comprising as main chain constituent units of formulae (XII) and/or (XIII): 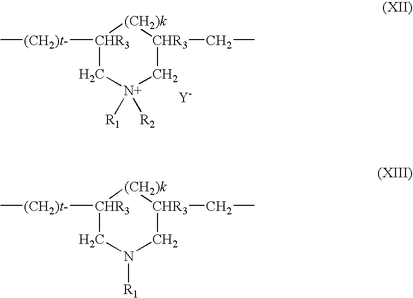
wherein k and t are integers ranging from 0 to 1, the sum k+t being 1; R₃ is chosen from a hydrogen atom and a methyl radical; R₁ and R₂, independently of each other, are chosen from C₁-C₆ alkyl groups, hydroxyalkyl groups, lower amidoalkyl groups comprising 1 to 5 carbon atoms, or R₁ and R₂ can form, together with the nitrogen atom to which they are attached, heterocyclic groups; and Y⁻ is an anion.

35. The cosmetic composition according to claim 34, wherein the at least one cationic polymer (iii) with a cationic charge density of greater than 4 meq./g is chosen from the polymers of formula (XII) wherein R₁ and R₂ are chosen from, independently of each other, methyl and ethyl, and R₃ is a hydrogen atom.

36. The cosmetic composition according to claim 35, wherein the at least one cationic polymer (iii) with a cationic charge density of greater than 4 meq./g is chosen from diallyldimethylammonium chloride homopolymers and copolymers of diallyldimethylammonium chloride and of acrylamide.

37. The cosmetic composition according to claim 1, wherein the at least one cationic polymer (iii) with a cationic charge density of greater than 4 meq./g is present in an amount ranging from 0.001% to 10% by weight, relative to the total weight of the composition.

38. The cosmetic composition according to claim 1, wherein the composition is a rinse-out or leave-in hair-conditioning composition.

39. The cosmetic composition according to claim 1, wherein the composition is a foaming detergent composition in a cosmetically acceptable medium:

40. A method of washing or caring for keratin materials, comprising applying a composition, in a cosmetically acceptable medium, wherein the composition comprises:
(i)—at least one cationic polymer, which is a produced by polymerization of a monomer mixture comprising:
a) at least one vinyl monomer substituted with at least one amino group, and
b) at least one hydrophobic nonionic vinyl monomer chosen from formula (I) and (II):
CH₂═C(X)Z, and (I)
CH₂═CH—OC(O)R; (II)
wherein:
X is chosen from a hydrogen atom and a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —SiR₃, —C(O)O(CH₂)ₓSiR₃, —C(O)NH(CH₂)ₓSiR₃, and —(CH₂)ₙSiR₃;
x is an integer ranging from 1 to 6;
each R independently is a C₁-C₃₀ alkyl group; and
c) at least one associative vinyl monomer,
(ii)—at least one amino silicone, and
(iii)—at least one cationic polymer other than the at least one cationic polymer (i) and the at least one amino silicone (ii) and having a cationic charge density of greater than or equal to 4 meq./g,
optionally followed by rinsing with water, after an optional leave-in time.
